(19) United States
(12) Patent Application Publication
(10) Pub. No.: US 2026/0042772 A1
(43) Pub. Date: Feb. 12, 2026

(54) FURO PYRIMIDINE DERIVATES

(71) Applicant: Merck Patent GmbH, Darmstadt (DE)

(72) Inventors: Shashank Kulkarni, Billerica, MA (US); Thomas Spangenberg, Aubonne (CH); Diego Gonzalez Cabrera, Cape Town (ZA); Nishanth Kandepedu, Cape Town (ZA); Thomas W. Von Geldern, Meyrin (CH); Gregory Basarab, Cape Town (ZA)

(73) Assignee: Merck Patent GmbH, Darmstadt (DE)

(21) Appl. No.: 19/101,914

(22) PCT Filed: Aug. 7, 2023
(86) PCT No.: PCT/EP2023/071763

§ 371 Date: Feb. 7, 2025

Related U.S. Application Data
(60) Provisional application No. 63/370,835, filed on Aug. 9, 2022.
(60) Provisional application No. 63/480,828, filed on Jan. 20, 2023.

(51) Int. Cl. C07D 491/048; A61K 31/519; A61K 31/5377; A61K 31/5386; A61K 31/541; C07D 519/00

(52) U.S. Cl. CPC C07D 491/048; A61K 31/519; A61K 31/5377; A61K 31/5386; A61K 31/541; C07D 519/00

(57) ABSTRACT

Novel compounds having valuable properties find application in the preparation of medicaments. In a method of their application, said novel compounds are employed as PI4K inhibitors and in the treatment or prevention of PI4K-related disorders such as protozoan infections like malaria and virus infections.

### BACKGROUND OF THE INVENTION

### Field of the Invention

The present invention relates to novel compounds having valuable properties, in particular those which can be used for the preparation of medicaments as well as methods of their use and manufacture. Said compounds are particularly useful as PI4K inhibitors and for the treatment or prevention of PI4K-related disorders such as protozoan infections like malaria and virus infections.

### Description of the Related Art

Malaria represents a major global health burden with an estimated 229 million new cases and nearly 409 000 deaths in 2019, mostly affecting young children and pregnant women (World Malaria Report 2020; World Health Organization: Geneva, Switzerland, 2020.). It is a vector-borne infectious disease caused by the hematoprotozoan parasite of genus Plasmodium (Phillips, M. A. et al., Malaria. Nat. Rev. Dis. Prim. 2017, 3, 17050). According to data from the World Health Organization (WHO), Plasmodium falciparum was responsible for the vast majority of malaria related morbidity and mortality in sub-Saharan Africa.

Currently, the WHO recommends artemisinin-based combination therapy (ACT). Additionally, vector control measures are key players in relieving the malarial burden. However, reports of emerging resistance toward ACTs (Dondorp, A. M et al., Artemisinin Resistance in Plasmodium falciparum Malaria. N. Engl. J. Med. 2009, 361, 455-467) illustrate the necessity of a new generation of drugs to combat resistance and improve standard of care for millions of affected patients. In recent years, research has identified novel druggable target structures, which can affect plasmodium viability. The availability of a limited number of validated drug targets such as dihydrofolate reductase, cytochrome c-oxidoreductases, and hemozoin formation are a promising starting point for the development of new anti-malaria compounds and emphasizes the need to expand chemical matter toward more efficacious drugs with novel modes of action and multistage antiparasitic activity.

Within this context, Plasmodium kinases are attractive targets for new generation anti-malarials as both protein and lipid kinases are involved in key signaling pathways at various stages of the parasite lifecycle and have had some level of genetic or phenotypic validation (Arendse, L. B. et al., Plasmodium Kinases as Potential Drug Targets for Malaria: Challenges and Opportunities, ACS Infect Dis. 2021, 7(3):518-534. doi: 10.1021/acsinfecdis.0c00724).

For example, lipid kinases are important in all stages of the Plasmodium lifecycle; this includes phosphatidylinositol-4-kinase (PI4K) that catalyzes the conversion of phosphatidylinositol (PI) to phosphatidylinositol-4-phosphate (PI4P). Phosphatidylinositol 4-kinase type III beta (PI4KIIIβ) is a ubiquitous eukaryotic enzyme that phosphorylates lipids to regulate intracellular signaling and trafficking. Imidazopyrazines are known inhibitors of PI4Ks. In blood stages of malaria, imidazopyrazines block a late step in parasite development by disrupting plasma membrane ingression around developing daughter merozoites. This likely stems from altered phosphatidylinositol 4-phosphate (PI4P) pools and disrupted Rab11A-mediated membrane trafficking. (McNamara, C. W. et al., Targeting Plasmodium PI(4)K to Eliminate Malaria. Nature 2013, 504 (7479), 248-253). Plasmodium PI4K is therefore important for signal transduction and membrane trafficking and has been shown to be a validated drug target for prevention, treatment, and elimination of malaria.

Several agents were recently reported as being Plasmodium PI4K inhibitors and the 2-aminopyridine MMV390048 had reached Phase Ila clinical trials (Paquet, T. et al., Antimalarial Efficacy of MMV390048, an Inhibitor of Plasmodium Phosphatidylinositol 4-Kinase. Sci. Transl. Med. 2017, 9 (387), 1-14) and further related compounds is shown below (PvPI4K, Pv=Plasmodium vivax).

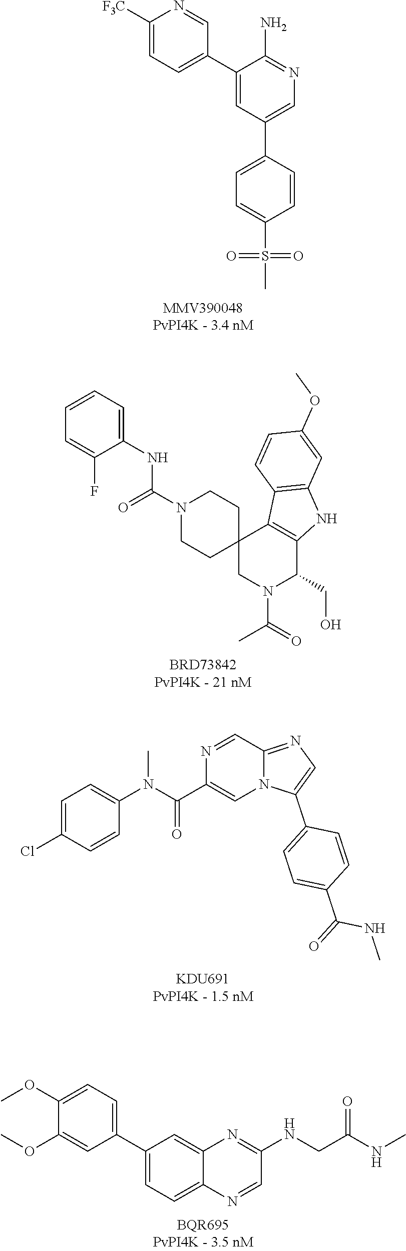

While PI4K has been identified as a useful target for protozoan infection treatment, human PI4K is also well-known to be hijacked by viruses. In particular human PI4KIIIβ, is an important host-target for viruses such as RNA viruses [PMID: 20510927; PMID: 33022924]. Therefore, PI4K inhibitors show great potential for the treatment of PI4K-related disorders such as virus or malaria infections.

WO 2012 025187 A1 discloses heterocyclic compounds useful as inhibitors of Syk that can be employed for the treatment of rheumatoid arthritis and/or systemic lupus.

WO 2013 117285 A1 discloses heterocyclic compounds useful as inhibitors of TBK1 and IKKε that can be employed for the treatment of cancer and inflammatory diseases.

WO 2013 124025 A1 discloses heterocyclic compounds useful as inhibitors of Syk that can be employed for the treatment of rheumatoid arthritis and/or systemic lupus.

WO 2017 003995 A1 discloses heterocyclic compounds useful as TBK/IKKε inhibitors.

WO 2011 086531 A1 and WO 2013 121387 A1 disclose the use of aminopyridine derivatives in the manufacture of medicaments for preventing or treating malaria. Specifically, the disclosure relates to aminopyridine derivatives useful for the inhibition of malaria parasite proliferation.

While many new compounds are in development, the need for a broader pallet of effective drugs targeting PI4K is high in order to allow for combination therapies to suppress the development of resistance to single compounds. Additionally, several lead structures, which have demonstrated acceptable PI4K inhibition require high dosages in order to be effective in vivo. The constant development and improvement of compounds is necessary to adapt biophysical properties of compounds to increase bio-availability as well as tolerability in clinical settings. Therefore, it has been the object of the present invention to overcome the disadvantages associated with the state-of-the-art as explained above and provide alternatives with high efficacy.

### SUMMARY OF THE INVENTION

The present invention provides compounds according to formula (I). Said compounds have been shown to be useful in the prevention and/or treatment of PI4K-related disorders such as malaria or viral infections and further relates to pharmaceutical compositions comprising said compounds. In particular compounds according to the present invention showed high selectivity in inhibition of Plasmodium PI4K, while affecting human PI4K to a lower extend, thereby providing drugs with potentially lower side effects due to human PI4K inhibition.

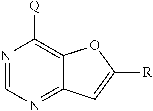

In another aspect, the present invention provides compounds of formula (I), which are suitable as PI4K inhibitors. Said compounds preferably inhibit Plasmodium PI4K and significantly reduce growth.

In certain embodiments, the present invention provides compounds of formula (I), which are selective PI4K inhibitors. In certain embodiments, the present invention provides compounds of formula (I), which are selective for Plasmodium PI4K.

In certain embodiments, the present invention provides compounds of formula (I), which inhibit human PI4K, more preferably human PI4KIIIβ.

In certain embodiments, the present invention provides compounds of formula (I), for use in the prevention and/or treatment of virus infections, most preferably virus infections caused by RNA viruses.

In further embodiments, the invention relates to pharmaceutical composition for use in the prevention and/or treatment of PI4K-related disorders, comprising at least one compound of formula (I).

In another aspect, the present invention provides methods for the treatment and/or prevention of malaria comprising administering a compound of formula (I). In another aspect, the present invention provides compounds which are able to modulate, especially inhibit the activity of PI4K in a disease state in mammals.

For convenience, certain terms employed in the specification, examples, and appended embodiments are collected here and provide definitions of the various chemical moieties that make up the compounds according to the invention and are intended to apply uniformly through-out the specification and embodiments, unless an otherwise expressly set out definition provides a broader definition.

The term “pharmaceutically acceptable salts or complexes” refers to salts or complexes of the compounds according to the invention. Examples of such salts include, but are not restricted, to base addition salts formed by reaction of compounds of the invention with organic or inorganic bases such as hydroxide, carbonate or bicarbonate of a metal cation such as those selected in the group consisting of alkali metals (sodium, potassium or lithium), alkaline earth metals (e.g. calcium or magnesium). Also comprised are salts which are formed from acid addition, salts formed with inorganic acids (e.g. hydrochloric acid, hydrobromic acid, sulfuric acid, phosphoric acid, nitric acid, and the like), as well as salts formed with organic acids such as acetic acid, oxalic acid, tartaric acid, succinic acid, malic acid, fumaric acid, maleic acid, ascorbic acid, benzoic acid, tannic acid, palmoic acid, alginic acid, polyglutamic acid, naphthalene sulfonic acid, naphthalene disulfonic acid, and poly-galacturonic acid.

“Pharmaceutically active compounds” refers to any compound that upon administration to the recipient, is capable of providing directly or indirectly, the activity disclosed herein. The term “indirectly” also encompasses prodrugs which may be converted to the active form of the drug via endogenous enzymes or metabolism. The prodrug is a derivative of the compounds according to the invention and presenting anti-malarial activity that has a chemically or metabolically decomposable group, and a compound that may be converted into a pharmaceutically active compound according to the invention in vivo by solvolysis under physiological conditions. The prodrug is converted into a compound according to the present invention by a reaction with an enzyme, gastric acid or the like under a physiological condition in the living body, e.g., by oxidation, reduction, hydrolysis or the like, each of which is carried out enzymatically. These compounds can be produced from compounds of the present invention according to well-known methods.

The term “solvates” of the compounds is taken to mean adductions of inert solvent molecules onto the compounds which form owing to their mutual attractive force. Solvates are, for example, mono- or dihydrates or alkoxides. The term “indirectly” also encompasses metabolites of compounds according to the invention.

The term “metabolite” refers to all molecules derived from any of the compounds according to the present invention in a cell or organism, preferably mammal.

The term “malaria” includes disease and conditions related to an infection with Plasmodium.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or relieving the disease, i.e., causing regression of the disease and/or its symptoms or conditions. The term “effective amount” includes “prophylaxis-effective amount” as well as “treatment-effective amount”.

The term “prophylaxis-effective amount” refers to a concentration of compound of this invention that is effective in inhibiting, decreasing the likelihood of the disease by malarial parasites, or preventing malarial infection or preventing the delayed onset of the disease by malarial parasites, when administered before infection, i.e., before, during and/or slightly after the exposure period to malarial parasites.

The term “prophylaxis” includes causal prophylaxis, i.e., antimalarial activity comprising preventing the pre-erythrocytic development of the parasite, suppressive prophylaxis, i.e. antimalarial activity comprising suppressing the development of the blood stage infection and terminal prophylaxis, i.e. antimalarial activity comprising suppressing the development of intra-hepatic stage infection. This term includes primary prophylaxis (i.e. preventing initial infection) where the antimalarial compound is administered before, during and/or after the exposure period to malarial parasites and terminal prophylaxis (i.e. to prevent relapses or delayed onset of clinical symptoms of malaria) when the antimalarial compound is administered towards the end of and/or slightly after the exposure period to malarial parasites but before the clinical symptoms. Further, this term includes suppression of dormant forms of the parasite in the liver (intrahepatic or pre-erythrocytic stage) as well as the activation and elimination of dormant forms (wake-up-and-kill concept). Typically, against P. falciparum infections, suppressive prophylaxis is used whereas against P. ovale, P. vivax or a combination of P. falciparum and P. vivax, terminal prophylaxis is used. Suppression of dormant stages is particularly useful against P. ovale and P. vivax.

The expression “effective amount” denotes the amount of a medicament or of a pharmaceutical active ingredient which causes in a tissue, system, animal or human a biological or medical response which is sought or desired, for example, by a researcher or physician.

Likewise, the term “treatment-effective amount” or “therapeutically effective amount” refers to an amount of compound which, compared with a corresponding subject who has not received this amount, has the following consequence: improved treatment, healing, prevention or elimination of a disease, syndrome, condition, complaint, disorder or side-effects or also the reduction in the advance of a disease, complaint or disorder.

The expression “treatment-effective amount” or “therapeutically effective amount” also encompasses the amounts which are effective for increasing normal physiological function and are necessary for effective treatment of a disease such as a malaria infection e.g. leading to a reduction in parasite numbers in blood following microscopic examination when administered after infection has occurred.

The expression “PI4K-related disorders” refer to disorders affected by PI4K interactions such as inhibition or overexpression of PI4K caused by for example pathogens, genetic predisposition, usage of PI4K for viral replication, as well as disorders treatable and/or preventable by inhibition of PI4K of the patient or of parasites such as Plasmodium. Examples for PI4K-related disorders are, but not limited to, viral infections such as infections caused by RNA viruses or protozoan infections such as malaria.

The term “subject” as used herein refers to mammals. For examples, mammals contemplated by the present invention include humans and the like.

The term “pharmaceutically acceptable carrier, adjuvant, or excipient” refers to a nontoxic carrier, adjuvant, or excipient that does not destroy the pharmacological activity of the compound with which it is formulated. Pharmaceutically acceptable carriers, adjuvants or excipient that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitory active metabolite or residue thereof.

A wavy line at the end of a bond line, generally appearing perpendicular to the bond line, has the same meaning as a wavy line that bisects a bond line.

### DETAILED DESCRIPTION OF THE INVENTION

The aim of the present invention was the development and identification of novel compounds useful for inhibition of PI4K and the treatment of PI4K-related disorders such as malaria or viral infections in order to extend, offer alternatives and improve limited treatment options for physicians and vets thereby ensuring highly effective treatments for patients.

Surprisingly, it has been discovered that compounds according to the present invention are inhibitors of PI4K, which is found in multiple organisms.

Therefore, the present invention relates to a compound according to formula (I)

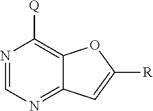

or a pharmaceutically acceptable solvate, salt, tautomer or stereoisomer thereof, wherein:
R denotes AR1 or HT1;
AR1 denotes phenyl, which is unsubstituted or substituted by
   1, 2 or 3 substituents independently selected from: Alk2, OAlk2, Hal, Cyc, CN and/or NO₂ (preferably Alk2, OAlk2, Hal and/or Cyc); and/or
   a substituent selected from a group comprising: A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
HT1 denotes a mono- or bicyclic saturated, unsaturated or aromatic heterocycle with 3 to 9 carbon atoms and 1, 2, 3 or 4 N, O and/or S atoms, wherein said heterocycle is unsubstituted or substituted by:
   1, 2 or 3 substituents independently selected from: Alk2, OAlk2, Hal, Cyc, CN, ═O and/or NO₂ (preferably Alk2, OAlk2, Hal and/or Cyc); and/or
   a substituent selected from a group comprising: A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyC, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCycl, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}) (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
Q denotes a structure according to formula (II)

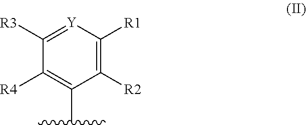

R¹ denotes AR2 or HT2;
R², R³ and R⁴ denote, independently from each other, H, Hal or CAlk2;
Y denotes CH, CHal, CAlk2, CCHal3 or N;
AR2 denotes phenyl, which is unsubstituted or substituted by
   1, 2 or 3 substituents independently selected from: Alk2, OAlk2, Hal, Cyc, CN and/or NO₂ (preferably Alk2, OAlk2, Hal, and/or Cyc); and/or
   a substituent selected from a group comprising:
      A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
HT2 denotes a mono- or bicyclic saturated, unsaturated or aromatic heterocycle with 3 to 9 carbon atoms and 1, 2, 3 or 4 N, O and/or S atoms, wherein said heterocycle is unsubstituted or substituted by
   1, 2 or 3 substituents independently selected from: Alk2, OAlk2, Hal, Cyc, CN, ═O and/or NO₂ (preferably Alk2, OAlk2, Hal, and/or Cyc); and/or
   a substituent selected from a group comprising:
      A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONR^{Cyc3}R^{Cyc4}, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyC, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
A denotes linear or branched alkyl having 1, 2, 3, 4, 5, or 6 carbon atoms, wherein:
   one or two non-adjacent CH₂ groups may be replaced by O, NAlk2 or NH; and/or
   1, 2, 3, 4 or 5 hydrogens may be replaced by Hal; and/or
   one hydrogen may be replaced by OH or NH₂ or a cyclic alkyl having 3, 4, 5 or 6 carbon atoms, which is mono- di or trisubstituted by Hal, OH, Alk2, NHAlk2, N(Alk2)₂ and/or NH₂;
Alk1 denotes linear or branched alkyl having 1, 2, 3, 4, 5, or 6 carbon atoms, wherein—one or two CH₂ groups may be replaced by O, NAlk2 or NH; and/or
   1 hydrogen may be replaced by OH, NHAlk2, N(Alk2)₂ or NH₂; and/or
   1, 2, 3, 4 or 5 hydrogens may be replaced by Hal;
Alk2 denotes linear or branched alkyl having 1 to 6 carbon atoms, wherein 1, 2, 3, 4 or 5 hydrogens may be replaced by Hal;
Aryl denotes phenyl, which is unsubstituted or mono-, di- or trisubstituted Hal, Alk2, OAlk2, OH, NH₂ or Cyc;
HetCyc1 denotes a mono- or bicyclic optionally bridged saturated or unsaturated 4- to 10-membered heterocycle having 1 or 2 heteroatoms selected from N, O, S and/or Si, wherein said heterocycle may be unsubstituted or mono- or disubstituted by Hal, OH, A, SO₂Alk2 and/or ═O;
Cyc denotes cyclic alkyl having 3 to 6 carbon atoms, wherein 1, 2 or 3 hydrogens may be replaced by Hal and 1 additional hydrogen may be replaced by Alk2, NH₂ and/or OH;
Hal denotes F or Cl;
HetAr1 denotes a mono- or bicyclic aromatic 4- to 12-membered heterocycle having 1, 2, 3 or 4 N, O and/or S atoms, said heterocycle being unsubstituted or mono- or disubstituted Hal, Alk2, SOAlk2, SO₂Alk2, OH or NH₂;
R^{a} and R^{b} denote each, independently from each other, H, Alk2 or Cyc;
   or
   R^{a} and R^{b} together represent —(CH₂)ₓ— with x=2, 3, 4 or 5, thus forming together with the carbon atom they are attached to a (3-, 4-, 5- or 6-membered) cycloalkyl ring;
R^{Cyc1} and R^{Cyc2} together form —(CH₂)ₓ— with x=3 or 4, thus forming together with the atoms they are attached to a (5- or 6-membered) ring, wherein 1, or 2H atoms, in —(CH₂)ₓ— may be independently replaced by Hal or Alk1;
R^{Cyc3} and R^{Cyc4} together form —(CH₂)ₓ— with x=3, 4 or 5, thus forming together with the nitrogen atom they are attached to a (4-, 5- or 6-membered) ring, wherein 1, or 2H atoms, in —(CH₂)ₓ— may be independently replaced by Hal or Alk1;
n denotes 0, 1 or 2 (preferably 0 or 1); and
m denotes 0 or 1 (preferably 0).

The compounds of formula (I) according to the present invention may—also depending on the nature of substituents they may bear—have one or more centers of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers”. It may be desirable to use a specific stereoisomer, e.g. one specific enantiomer or diastereomer of a certain compound. In these cases, a compound according to the present invention obtained as a racemate or even intermediates thereof—may be separated into the stereoisomeric (enantiomeric, diastereoisomeric) compounds by chemical or physical measures known to the person skilled in the art. The compounds of the invention which have one or more centers of chirality and which occur as racemates or as mixtures of enantiomers or diastereoisomers can for example be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes.

Examples of compounds according to the present invention, which have a stereogenic center are:

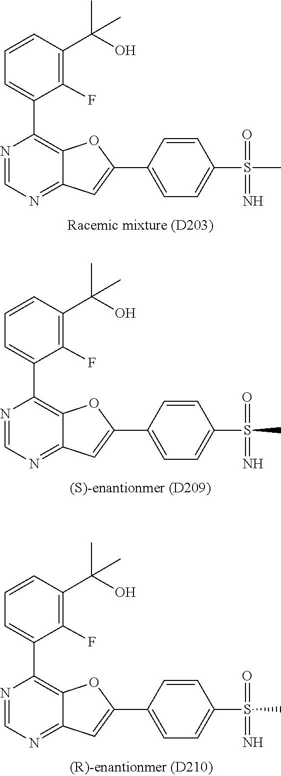 [SMILES: CC(C)(O)c1cccc(-c2ncnc3cc(-c4ccc(S(C)(=N)=O)cc4)oc23)c1F ; CC(C)(O)c1cccc(-c2ncnc3cc(-c4ccc([S@@](C)(=N)=O)cc4)oc23)c1F ; CC(C)(O)c1cccc(-c2ncnc3cc(-c4ccc([S@](C)(=N)=O)cc4)oc23)c1F]

In this specific example the sulfur atom represents the stereocenter, in other examples of the present invention the compounds may of course have other or additional stereocenters located at a different atom e.g. at a carbon atom. As shown above, in case a compound with one or more stereocenter(s) is shown without specifying the stereoconfiguration at the stereocenter(s) this refers to a mixture of the corresponding stereoisomers.

In a certain embodiment of the present invention residue R of a compound according to formula (I) denotes a structure according to formula (IV), (V), (Va) or (VI)

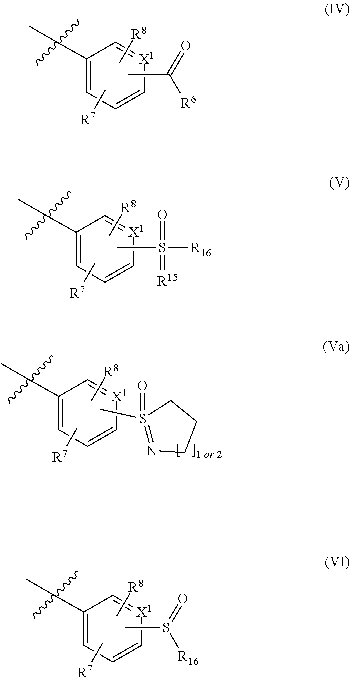

wherein
R⁶ denotes OH, A or Cyc (preferably OH, cyclopropyl, OCH₃, OCF₃, OCHF₂, CH₃, C₂H₅, CH₂F, CHF₂, OC₂H₅, OiPr, OtBu, NH₂, NHCH₃, N(CH₃)₂, N(C₂H₅)₂, N(iPr)₂, N(CH₃)(nPr) or N(CH₃)(tBu)) or a substituent according to formula (VII) to (X)

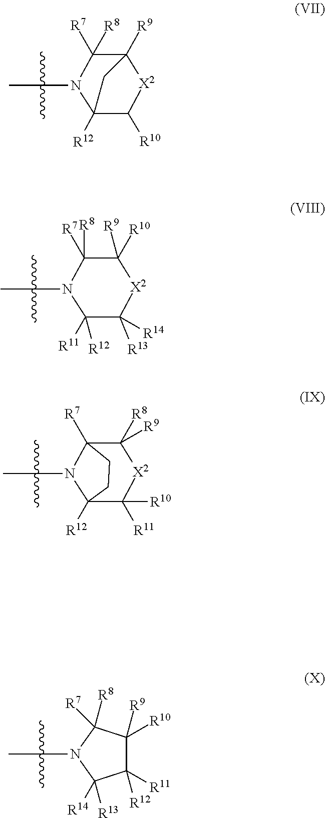

wherein
R⁷, R⁸, R⁹, R¹⁰, R¹¹, R¹², R¹³ and R¹⁴ denote each, independently from each other, H, OH, Hal, CH₃, C₂H₅, CHal₃, OCH₃, OCHal₃, OCHal₂, OCH₂Hal, CH₂Hal and/or CHHal₂;
R¹⁵ denotes NR¹⁷ or O;
R¹⁶ denotes A or Cyc;
R¹⁷ denotes H, Alk1 or cyclic alkyl having 3 to 6 carbon atoms, wherein 1, 2 or 3 hydrogens of said cyclic alkyl group may be replaced by Hal;
X¹ denotes N or CH; and
X² denotes NH, NAlk1 or O.

For the avoidance of doubt, residues R⁷, R⁸ COR⁶, SOR¹⁶(NR¹⁵) and SOR¹⁶ of formulas (IV), (V), and (VI) and the cyclic S-residue of formula (Va) as shown above may be attached to each of the carbon atoms of the aromatic ring. In important embodiments residues COR⁶, SOR¹⁶(NR¹⁵) and SOR¹⁶ are attached in para-position to the carbon atom, which connects residue R with the annulated ring system (the furo pyrimidine residue), as shown in formulas (IVb)-(VIb) below,

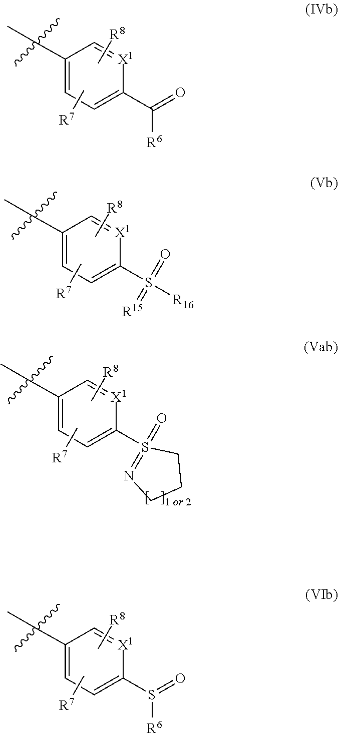

and residues R⁷ and R⁸ are each preferably attached in ortho- or meta-position to the carbon atom which connects residue R with the annulated ring system.

Another particular embodiment concerns a compound according to formula (I) as defined above, wherein Q denotes a residue according to formula (XI)

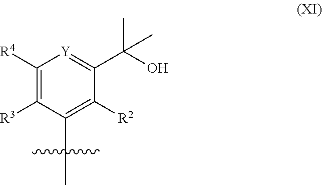

wherein
Y denotes N or CH;
and one or two of the residues R², R³ and R⁴ independently represent, Hal, CH₃, CHal₃, OCH₃, OCHal₃, OCHHal₂, OCH₂Hal, CH₂Hal and/or CHHal₂ and the remaining of said residue(s) represent H.

In such an embodiment R² and R⁴ preferably represent independently from one another, a residue selected from F, CH₃, CF₃, OCH₃, OCF₃, OCHF₂, OCH₂F, CH₂F and/or CHF₂ and R³ denotes H. In another important embodiment of a residue according to formula (XI) as described above R² represents, a residue selected from F, CH₃, CF₃, OCH₃, OCF₃, OCHF₂, OCH₂F, CH₂F and/or CHF₂ and R³ and R⁴ denote H. In further important embodiment of said residue R⁴ represents, a residue selected from F, CH₃, CF₃, OCH₃, OCF₃, OCHF₂, OCH₂F, CH₂F and/or CHF₂ and R³ and R² denote H.

A further particular embodiment concerns a compound according to formula (I), wherein Z denotes N.

In the context of the present invention “hydroxyalkyl” represents a linear or branched hydrocarbon residue with 1, 2, 3, 4, 5 or 6 carbon atoms (preferably 1, 2, 3 or 4 carbon atoms), which is substituted with one or two (preferably one) hydroxy groups. Examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxypropan-2-yl, 1-hydroxyethyl, 2-hydroxy2-methylpropyl and the like.

Throughout the invention, all residues which occur more than once may be identical or different, i.e. are independent of one another. For example in “(CR^{a}R^{b})ₙCONH(CR^{a}R^{b})ₘHetCyc1” or “(CR^{a}R^{b})ₙSO₂NA₂” each instance of R^{a}, R^{b} and A may have a different meanig (within the scope of the corresponding definition).

In particular important embodiments A represents a linear or branched alkyl group having 1, 2, 3 or 4 carbon atoms, wherein (as applicable) one or two non-adjacent CH₂ groups may be replaced by O, NCH₃, NC₂H₅, NiPr or NH; and/or 1, 2, 3, 4 or 5 hydrogens may be replaced by Hal on/or one hydrogen may be replaced by OH, NH₂ or a cyclic alkyl group having 3, 4, 5, or 6 carbon atoms, wherein said cyclic alkyl group may be mono or disubstituted by Hal, Alk2 NHAlk2 (NAlk2)₂ and/or NH₂.

AR1 denotes preferably 3,4,5-trimethoxyphenyl or phenyl, which is
substituted by one or two residue(s) selected from Hal, CH₃, CHal₃ and/or OCH₃; and/or
one residue selected from: (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, ((CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA or (CR^{a}R^{b})ₙPOA₂.

HT1 denotes preferably pyridinyl, pyrimidinyl, pyrazinyl, pyridazinyl, indolyl, isoindolyl, benzofuranyl, benzothiophenyl, isoindolyl, benzoimidazoyl, indazolyl (most preferably pyridinyl) or one of following residues:

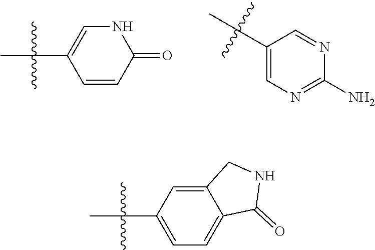 [SMILES: *c1ccc(=O)[nH]c1 ; *c1cnc(N)nc1 ; *c1ccc2c(c1)CNC2=O]

each of said residues, independently from one another unsubstituted or substituted by (following optional substituents, which may be attached to a carbon atom or another atom under the provisio that a proper valency of said atom results, are not shown in the residues depicted above):
1, 2 or 3 substituents independently selected from: A, Hal; and/or
a substituent selected from a group comprising: NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyC, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA, (CR^{a}R^{b})ₙPOA₂, and an azaspirocycle, which is unsubstituted or monosubstituted by at least one Hal, Alk2 or OAlk2 group.
HT2 denotes preferably pyridinyl, pyrimidinyl, pyrazinyl, pyridazinyl, indolyl, isoindolyl, benzofuranyl, benzothiophenyl, isoindolyl, benzoimidazoyl, indazolyl (most preferably pyridinyl) or one of following residues:

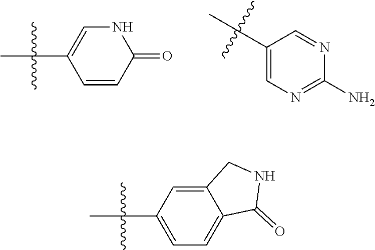 [SMILES: *c1ccc(=O)[nH]c1 ; *c1cnc(N)nc1 ; *c1ccc2c(c1)CNC2=O]

each of said residues, independently from one another unsubstituted or substituted by (following optional substituents, which may be attached to a carbon atom or another atom under the provisio that a proper valency of said atom results, are not shown in the residues depicted above):
   1, 2 or 3 substituents independently selected from: A, Hal; and/or
   a substituent selected from a group comprising:
      NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA, (CR^{a}R^{b})ₙPOA₂, and an azaspirocycle, which is unsubstituted or monosubstituted by at least one Hal, Alk2 or OAlk2 group.

Cyc denotes preferably cyclopropyl, cyclobutyl, cyclopentyl, or cyclohexyl.

In a specific embodiment of the present invention residue R of a compound according to formula (I) denotes a structure according to formula (IV)

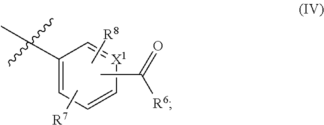

wherein
R⁶ is Alk1, Alk2, —OH, —CH₃, —OCH₃, —OC(CH₃)₃, —N(CH₃)₂,

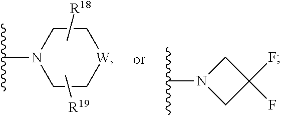

W is O, —NCH₃, NR¹⁸, or CR¹⁸R¹⁹;
R⁷, R⁸ is each independently selected from —H or Hal;
R¹⁸, R¹⁹ is each independently selected from —H, —CH₃, or Alk1;
X¹ is CR⁷ or N.

In this specific embodiment of the present invention residue Q of the compound denotes a structure according to formula (II) above, wherein
R¹ is H, Hal,

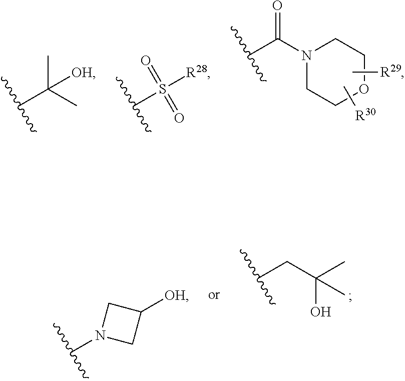

R², R³, R⁴ is each independently selected from H or Hal;
R²⁸ is Alk1, Alk2, —NH₂,

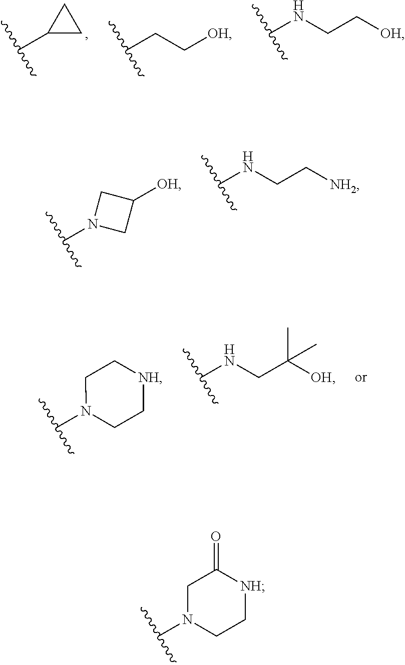

R²⁹, R³⁰ is each independently selected from —H or —CH₃; and
Y is N, CH, or CHal.

Further, in a preferred embodiment of the present invention,
R⁶ is —CH₃, —OH, —N(CH₃)₂;

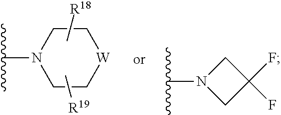

W is O;
R⁷, R⁸ is H;
R¹⁸, R¹⁹ is each independently selected from —H or —CH₃; and
X¹ is CH or N.

Further, in a preferred embodiment of the present invention,
R⁶ is

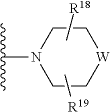

and R¹ is

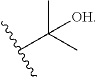 [SMILES: *C(C)(C)O]

In a specific embodiment of the present invention residue R of a compound according to formula (I) denotes a structure according to formula (V)

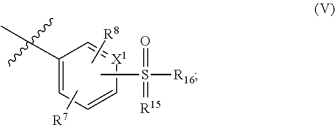

wherein
X¹ is CR⁷ or N;
R⁷, R⁸ is each independently selected from H, Hal, or CHal₃;
R¹⁵ is O or NH;
R¹⁶ is H, —CH₃, —NH₂, —N(CH₃)₂, or Alk1.

In this specific embodiment of the present invention residue Q of the compound denotes a structure according to formula (II) above, wherein
R¹ is H, Hal,

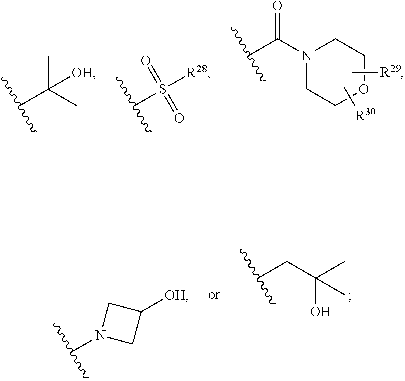

R², R³, R⁴ is each independently selected from H or Hal;
R²⁹ is Alk1, Alk2, —NH₂,

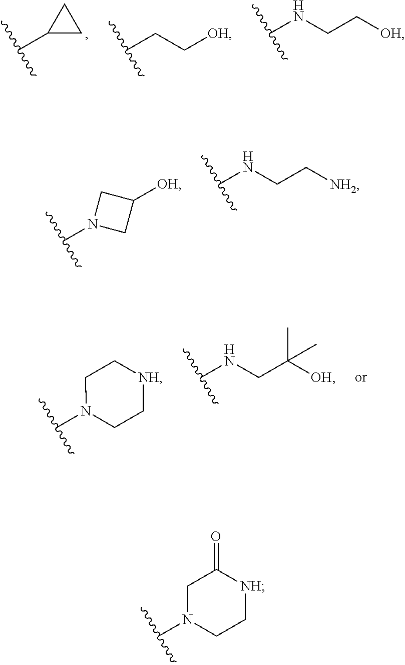

R²⁹, R³⁰ is each independently selected from —H or —CH₃; and
Y is N, CH, or CHal.

Further, in a preferred embodiment of the present invention,
R¹ is selected from H, Hal, or

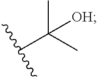 [SMILES: *C(C)(C)O]

R², R³, R⁴, R⁷, R⁸ is each independently selected from H or Hal;
R¹⁶ is —CH₃ or Alk1;
X¹ is CH; and
Y is CH or CHal.

Further, in a preferred embodiment of the present invention,
R¹ is

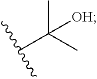 [SMILES: *C(C)(C)O]

R², R⁷, R⁸ is each independently selected from H or Hal;
R³, R⁴ is H;
R¹⁵ is NH;
R¹⁶ is —CH₃ or Alk1;
X¹ is CH; and
Y is CH.

Further, in a preferred embodiment of the present invention, the SOR¹⁵R¹⁶ group of formula V is ortho to the linkage of formula I. In some embodiments R³ and R⁴ are H. In some embodiments X¹ is CH.

Further, in a preferred embodiment of the present invention,
R¹ is

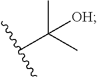 [SMILES: *C(C)(C)O]

R², R⁷, R⁸ is each independently selected from H or Hal;
R³, R⁴ is H;
R¹⁵ is NH;
R¹⁶ is —CH₃;
X¹ is CH; and
Y is CH.

Further, in a very specific embodiment of the present invention, the compound is

| | |
|---|---|
| D203 | (4-(4-(2-fluoro-3-(2-hydroxypropan-2-yl)phenyl)furo[3,2- |
| | d]pyrimidin-6-yl)phenyl)(imino)(methyl)-λ6-sulfanone. |

Further, in a very specific embodiment of the present invention, the compound is

| | |
|---|---|
| D209 | (S)-(4-(4-(2-fluoro-3-(2-hydroxypropan-2-yl)phenyl)furo[3,2- |
| | d]pyrimidin-6-yl)phenyl)(imino)(methyl)-λ⁶-sulfanone. |

Further, in a very specific embodiment of the present invention, the compound is

| | |
|---|---|
| D210 | (R)-(4-(4-(2-fluoro-3-(2-hydroxypropan-2-yl)phenyl)furo[3,2- |
| | d]pyrimidin-6-yl)phenyl)(imino)(methyl)-λ⁶-sulfanone. |

In a specific embodiment of the present invention residue R of a compound according to formula (I) denotes a one of the structures below

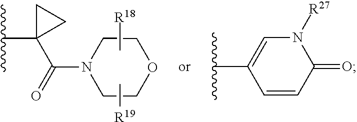

wherein
R¹⁸, R¹⁹ is each independently selected from H, —CH₃, or Alk1; and
R²⁷ is —CH₃ or —C((CH₃)₂OH).

In this specific embodiment of the present invention residue Q of the compound denotes a structure according to formula (II) above, wherein
R¹ is H, Hal,

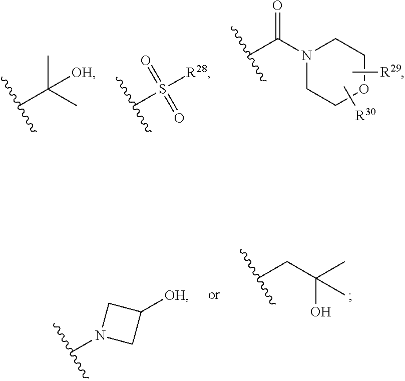

R², R³, R⁴ is each independently selected from H or Hal;
R²⁸ is Alk1, Alk2, —NH₂,

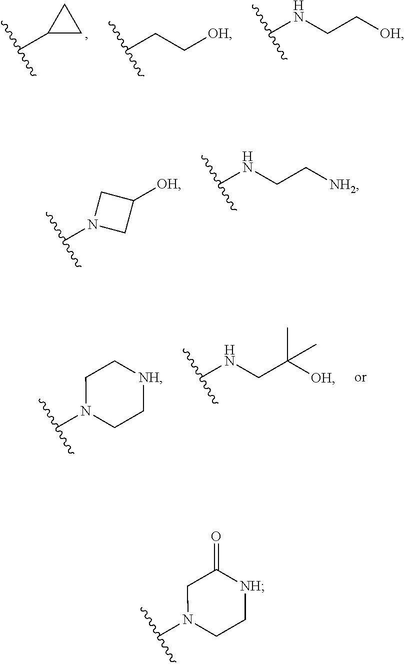

R²⁹, R³⁰ is each independently selected from H or CH₃; and
Y is N, CH, or CHal.

In some embodiments, compounds according to the present invention are selected from the group of:

| Comp. | Structure and/or Formula | Name |
|---|---|---|
| C74 | 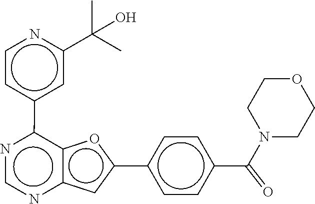 | (4-{4-[2-(1-Hydroxy-1- methyl-ethyl)-pyridin-4-yl]- furo[3,2-d]pyrimidin-6-yl}- phenyl)-morpholin-4-yl- methanone |
| C75 | 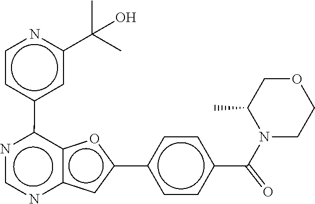 | (4-{4-[2-(1-Hydroxy-1- methyl-ethyl)-pyridin-4-yl]- furo[3,2-d]pyrimidin-6-yl}- phenyl)-((R)-3-methyl- morpholin-4-yl)-methanone |
| C76 | 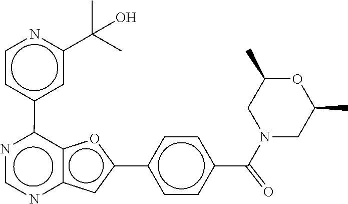 | 2-[4-(6-{4-[(2R,6S)-2,6- dimethylmorpholine-4- carbonyl]phenyl}furo[3,2- d]pyrimidin-4-yl)pyridin-2- yl]propan-2-ol |
| C77 | 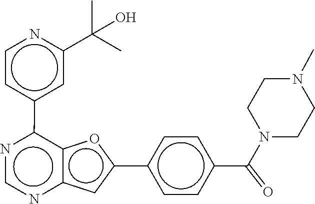 | 2-(4-{6-[4-(4- methylpiperazine-1- carbonyl)phenyl]furo[3,2- d]pyrimidin-4-yl}pyridin-2- yl)propan-2-ol |
| D22 | 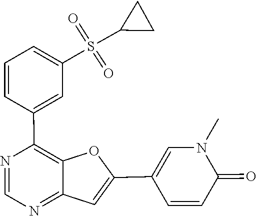 [SMILES: Cn1cc(-c2cc3ncnc(-c4cccc(S(=O)(=O)C5CC5)c4)c3o2)ccc1=O] | 5-(4-(3- (cyclopropylsulfonyl)phenyl) furo[3,2-d]pyrimidin-6-yl)- 1-methylpyridin-2(1H)-one |
| D149 | 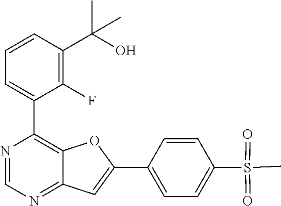 [SMILES: CC(C)(O)c1cccc(-c2ncnc3cc(-c4ccc(S(C)(=O)=O)cc4)oc23)c1F] | 2-(2-fluoro-3-(6-(4- (methylsulfonyl)phenyl)furo [3,2-d]pyrimidin-4- yl)phenyl)propan-2-ol |
| D150 | 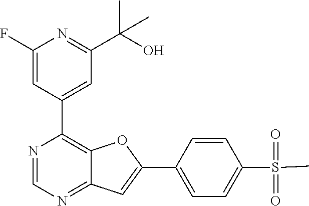 [SMILES: CC(C)(O)c1cc(-c2ncnc3cc(-c4ccc(S(C)(=O)=O)cc4)oc23)cc(F)n1] | 2-(6-fluoro-4-(6-(4- (methylsulfonyl)phenyl)furo [3,2-d]pyrimidin-4- yl)pyridin-2-yl)propan-2-ol |
| D159 | 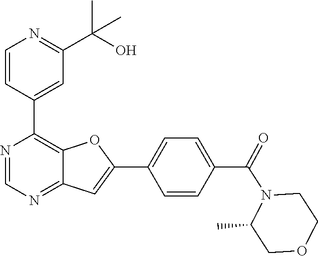 [SMILES: C[C@H]1COCCN1C(=O)c1ccc(-c2cc3ncnc(-c4ccnc(C(C)(C)O)c4)c3o2)cc1] | (S)-(4-(4-(2-(2- hydroxypropan-2- yl)pyridin-4-yl)furo[3,2- d]pyrimidin-6-yl)phenyl)(3- methylmorpholino) methanone |
| D160 | 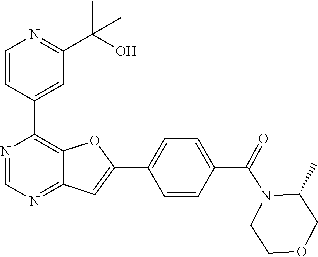 [SMILES: C[C@@H]1COCCN1C(=O)c1ccc(-c2cc3ncnc(-c4ccnc(C(C)(C)O)c4)c3o2)cc1] | (R)-(4-(4-(2-(2- hydroxypropan-2- yl)pyridin-4-yl)furo[3,2- d]pyrimidin-6-yl)phenyl)(3- methylmorpholino) methanone |
| D161 | 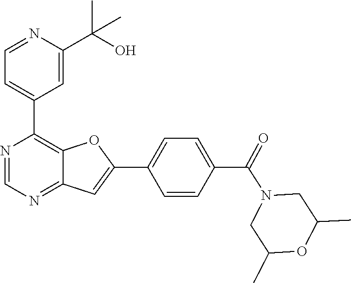 [SMILES: CC1CN(C(=O)c2ccc(-c3cc4ncnc(-c5ccnc(C(C)(C)O)c5)c4o3)cc2)CC(C)O1] | (2,6- dimethylmorpholino)(4-(4- (2-(2-hydroxypropan-2- yl)pyridin-4-yl)furo[3,2- d]pyrimidin-6- yl)phenyl)methanone |
| D162 | 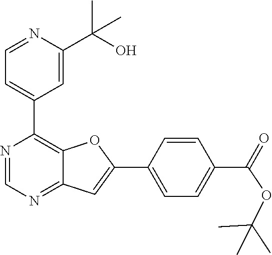 [SMILES: CC(C)(C)OC(=O)c1ccc(-c2cc3ncnc(-c4ccnc(C(C)(C)O)c4)c3o2)cc1] | tert-butyl 4-(4-(2-(2- hydroxypropan-2- yl)pyridin-4-yl)furo[3,2- d]pyrimidin-6-yl)benzoate |
| D163 | 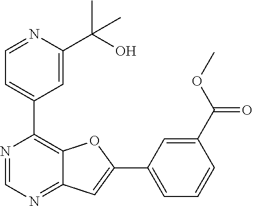 [SMILES: COC(=O)c1cccc(-c2cc3ncnc(-c4ccnc(C(C)(C)O)c4)c3o2)c1] | methyl 3-(4-(2-(2- hydroxypropan-2- yl)pyridin-4-yl)furo[3,2- d]pyrimidin-6-yl)benzoate |
| D164 | 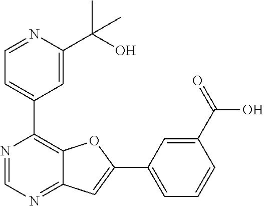 [SMILES: CC(C)(O)c1cc(-c2ncnc3cc(-c4cccc(C(=O)O)c4)oc23)ccn1] | 3-(4-(2-(2-hydroxypropan- 2-yl)pyridin-4-yl)furo[3,2- d]pyrimidin-6-yl)benzoic acid |
| D165 | 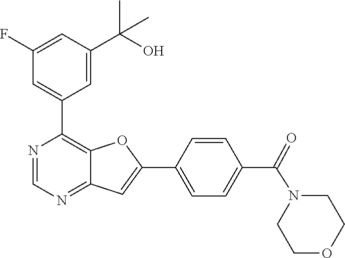 [SMILES: CC(C)(O)c1cc(F)cc(-c2ncnc3cc(-c4ccc(C(=O)N5CCOCC5)cc4)oc23)c1] | (4-(4-(3-fluoro-5-(2- hydroxypropan-2- yl)phenyl)furo[3,2- d]pyrimidin-6- yl)phenyl)(morpholino) methanone |
| D171 | 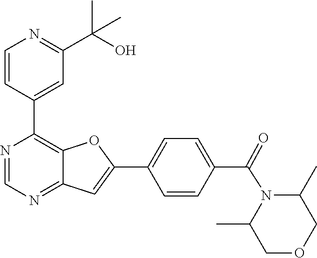 [SMILES: CC1COCC(C)N1C(=O)c1ccc(-c2cc3ncnc(-c4ccnc(C(C)(C)O)c4)c3o2)cc1] | (3,5- dimethylmorpholino)(4-(4- (2-(2-hydroxypropan-2- yl)pyridin-4-yl)furo[3,2- d]pyrimidin-6- yl)phenyl)methanone |
| D172 | 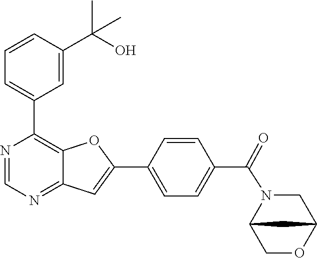 [SMILES: CC(C)(O)c1cccc(-c2ncnc3cc(-c4ccc(C(=O)N5C[C@H]6C[C@@H]5CO6)cc4)oc23)c1] | ((1R,4R)-2-oxa-5- azabicyclo[2.2.1]heptan-5- yl)(4-(4-(3-(2- hydroxypropan-2- yl)phenyl)furo[3,2- d]pyrimidin-6- yl)phenyl)methanone |
| D173 | 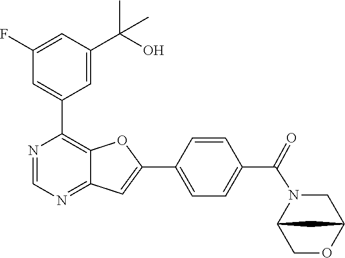 [SMILES: CC(C)(O)c1cc(F)cc(-c2ncnc3cc(-c4ccc(C(=O)N5C[C@H]6C[C@@H]5CO6)cc4)oc23)c1] | ((1R,4R)-2-oxa-5- azabicyclo[2.2.1]heptan-5- yl)(4-(4-(3-fluoro-5-(2- hydroxypropan-2- yl)phenyl)furo[3,2- d]pyrimidin-6- yl)phenyl)methanone |
| D174 | 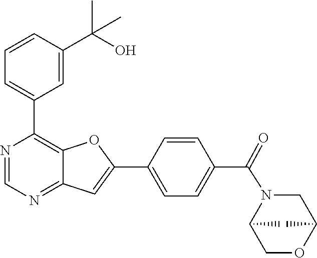 [SMILES: CC(C)(O)c1cccc(-c2ncnc3cc(-c4ccc(C(=O)N5C[C@@H]6C[C@H]5CO6)cc4)oc23)c1] | ((1S,4S)-2-oxa-5- azabicyclo[2.2.1]heptan-5- yl)(4-(4-(3-(2- hydroxypropan-2- yl)phenyl)furo[3,2- d]pyrimidin-6- yl)phenyl)methanone |
| D175 | 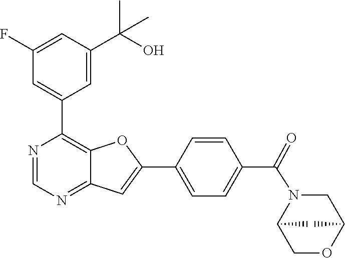 [SMILES: CC(C)(O)c1cc(F)cc(-c2ncnc3cc(-c4ccc(C(=O)N5C[C@@H]6C[C@H]5CO6)cc4)oc23)c1] | ((1S,4S)-2-oxa-5- azabicyclo[2.2.1]heptan-5- yl)(4-(4-(3-fluoro-5-(2- hydroxypropan-2- yl)phenyl)furo[3,2- d]pyrimidin-6- yl)phenyl)methanone |
| D176 | 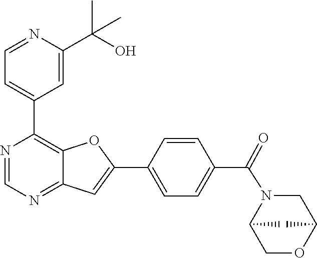 [SMILES: CC(C)(O)c1cc(-c2ncnc3cc(-c4ccc(C(=O)N5C[C@@H]6C[C@H]5CO6)cc4)oc23)ccn1] | ((1S,4S)-2-oxa-5- azabicyclo[2.2.1]heptan-5- yl)(4-(4-(2-(2- hydroxypropan-2- yl)pyridin-4-yl)furo[3,2- d]pyrimidin-6- yl)phenyl)methanone |
| D177 | 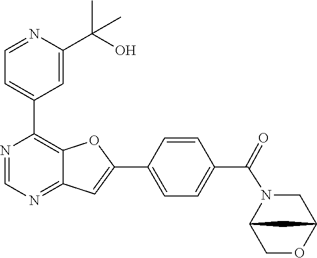 [SMILES: CC(C)(O)c1cc(-c2ncnc3cc(-c4ccc(C(=O)N5C[C@H]6C[C@@H]5CO6)cc4)oc23)ccn1] | ((1R,4R)-2-oxa-5- azabicyclo[2.2.1]heptan-5- yl)(4-(4-(2-(2- hydroxypropan-2- yl)pyridin-4-yl)furo[3,2- d]pyrimidin-6- yl)phenyl)methanone |
| D183 | 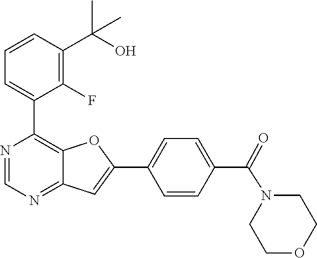 [SMILES: CC(C)(O)c1cccc(-c2ncnc3cc(-c4ccc(C(=O)N5CCOCC5)cc4)oc23)c1F] | (4-(4-(2-fluoro-3-(2- hydroxypropan-2- yl)phenyl)furo[3,2- d]pyrimidin-6- yl)phenyl)(morpholino) methanone |
| D191 | 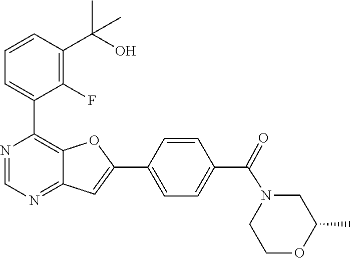 [SMILES: C[C@H]1CN(C(=O)c2ccc(-c3cc4ncnc(-c5cccc(C(C)(C)O)c5F)c4o3)cc2)CCO1] | (S)-(4-(4-(2-fluoro-3-(2- hydroxypropan-2- yl)phenyl)furo[3,2- d]pyrimidin-6-yl)phenyl)(2- methylmorpholino) methanone |
| D192 | 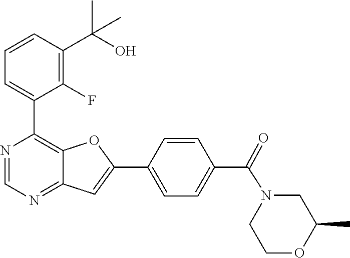 [SMILES: C[C@@H]1CN(C(=O)c2ccc(-c3cc4ncnc(-c5cccc(C(C)(C)O)c5F)c4o3)cc2)CCO1] | (R)-(4-(4-(2-fluoro-3-(2- hydroxypropan-2- yl)phenyl)furo[3,2- d]pyrimidin-6-yl)phenyl)(2- methylmorpholino) methanone |
| D193 | 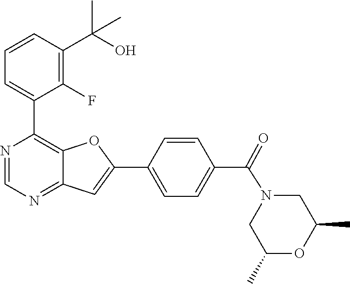 [SMILES: C[C@@H]1CN(C(=O)c2ccc(-c3cc4ncnc(-c5cccc(C(C)(C)O)c5F)c4o3)cc2)C[C@@H](C)O1] | ((2R,6R)-2,6- dimethylmorpholino)(4-(4- (2-fluoro-3-(2- hydroxypropan-2- yl)phenyl)furo[3,2- d]pyrimidin-6- yl)phenyl)methanone |
| D194 | 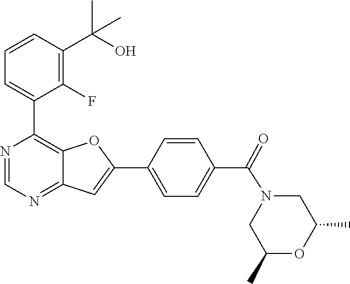 [SMILES: C[C@H]1CN(C(=O)c2ccc(-c3cc4ncnc(-c5cccc(C(C)(C)O)c5F)c4o3)cc2)C[C@H](C)O1] | ((2S,6S)-2,6- dimethylmorpholino)(4-(4- (2-fluoro-3-(2- hydroxypropan-2- yl)phenyl)furo[3,2- d]pyrimidin-6- yl)phenyl)methanone |
| D195 | 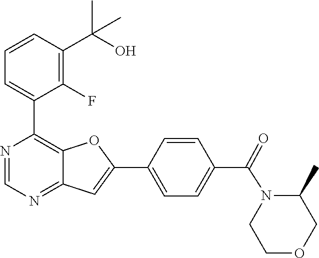 [SMILES: C[C@H]1COCCN1C(=O)c1ccc(-c2cc3ncnc(-c4cccc(C(C)(C)O)c4F)c3o2)cc1] | (S)-(4-(4-(2-fluoro-3-(2- hydroxypropan-2- yl)phenyl)furo[3,2- d]pyrimidin-6-yl)phenyl)(3- methylmorpholino) methanone |
| D196 | 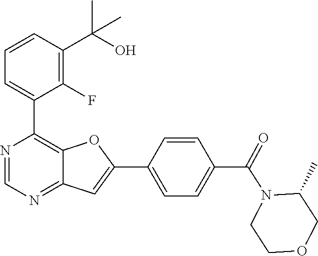 [SMILES: C[C@@H]1COCCN1C(=O)c1ccc(-c2cc3ncnc(-c4cccc(C(C)(C)O)c4F)c3o2)cc1] | (R)-(4-(4-(2-fluoro-3-(2- hydroxypropan-2- yl)phenyl)furo[3,2- d]pyrimidin-6-yl)phenyl)(3- methylmorpholino) methanone |
| D197 | 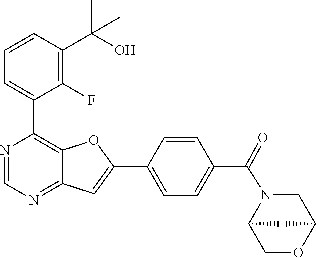 [SMILES: CC(C)(O)c1cccc(-c2ncnc3cc(-c4ccc(C(=O)N5C[C@@H]6C[C@H]5CO6)cc4)oc23)c1F] | ((1S,4S)-2-oxa-5- azabicyclo[2.2.1]heptan-5- yl)(4-(4-(2-fluoro-3-(2- hydroxypropan-2- yl)phenyl)furo[3,2- d]pyrimidin-6- yl)phenyl)methanone |
| D201 | 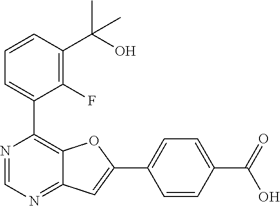 [SMILES: CC(C)(O)c1cccc(-c2ncnc3cc(-c4ccc(C(=O)O)cc4)oc23)c1F] | 4-(4-(2-fluoro-3-(2- hydroxypropan-2- yl)phenyl)furo[3,2- d]pyrimidin-6-yl)benzoic acid |
| D203 | 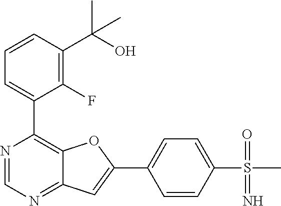 [SMILES: CC(C)(O)c1cccc(-c2ncnc3cc(-c4ccc(S(C)(=N)=O)cc4)oc23)c1F] | (4-(4-(2-fluoro-3-(2- hydroxypropan-2- yl)phenyl)furo[3,2- d]pyrimidin-6- yl)phenyl)(imino)(methyl)- λ6-sulfanone |
| D204 | 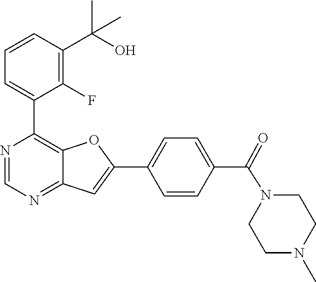 [SMILES: CN1CCN(C(=O)c2ccc(-c3cc4ncnc(-c5cccc(C(C)(C)O)c5F)c4o3)cc2)CC1] | (4-(4-(2-fluoro-3-(2- hydroxypropan-2- yl)phenyl)furo[3,2- d]pyrimidin-6-yl)phenyl)(4- methylpiperazin-1- yl)methanone |
| D207 | 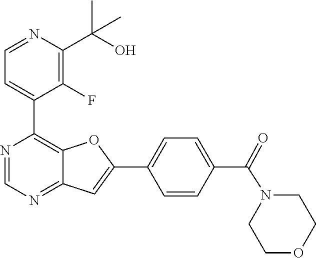 [SMILES: CC(C)(O)c1nccc(-c2ncnc3cc(-c4ccc(C(=O)N5CCOCC5)cc4)oc23)c1F] | (4-(4-(3-fluoro-2-(2- hydroxypropan-2- yl)pyridin-4-yl)furo[3,2- d]pyrimidin-6- yl)phenyl)(morpholino) methanone |
| D208 | 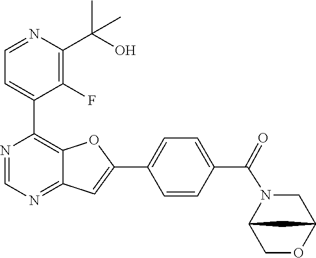 [SMILES: CC(C)(O)c1nccc(-c2ncnc3cc(-c4ccc(C(=O)N5C[C@H]6C[C@@H]5CO6)cc4)oc23)c1F] | ((1R,4R)-2-oxa-5- azabicyclo[2.2.1]heptan-5- yl)(4-(4-(3-fluoro-2-(2- hydroxypropan-2- yl)pyridin-4-yl)furo[3,2- d]pyrimidin-6- yl)phenyl)methanone |
| D209 | 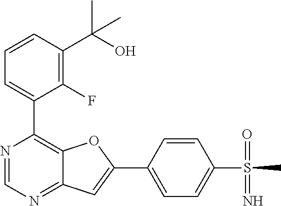 [SMILES: CC(C)(O)c1cccc(-c2ncnc3cc(-c4ccc([S@@](C)(=N)=O)cc4)oc23)c1F] | (S)-(4-(4-(2-fluoro-3-(2- hydroxypropan-2- yl)phenyl)furo[3,2- d]pyrimidin-6- yl)phenyl)(imino)(methyl)- λ⁶-sulfanone |
| D210 | 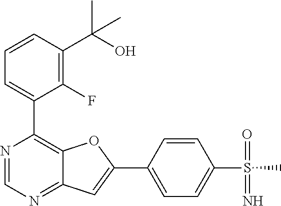 [SMILES: CC(C)(O)c1cccc(-c2ncnc3cc(-c4ccc([S@](C)(=N)=O)cc4)oc23)c1F] | (R)-(4-(4-(2-fluoro-3-(2- hydroxypropan-2- yl)phenyl)furo[3,2- d]pyrimidin-6- yl)phenyl)(imino)(methyl)- λ⁶-sulfanone |
| D211 | 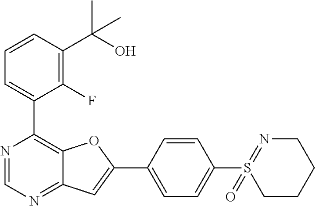 [SMILES: CC(C)(O)c1cccc(-c2ncnc3cc(-c4ccc(S5(=O)=NCCCC5)cc4)oc23)c1F] | 1-(4-(4-(2-fluoro-3-(2- hydroxypropan-2- yl)phenyl)furo[3,2- d]pyrimidin-6-yl)phenyl)- 3,4,5,6-tetrahydro-1,2- thiazine 1-oxide |
| D212 | 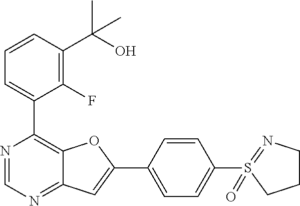 [SMILES: CC(C)(O)c1cccc(-c2ncnc3cc(-c4ccc(S5(=O)=NCCC5)cc4)oc23)c1F] | 1-(4-(4-(2-fluoro-3-(2- hydroxypropan-2- yl)phenyl)furo[3,2- d]pyrimidin-6-yl)phenyl)- 4,5-dihydro-3H-isothiazole 1-oxide |
| D213 | 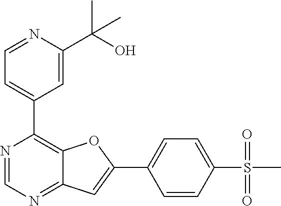 [SMILES: CC(C)(O)c1cc(-c2ncnc3cc(-c4ccc(S(C)(=O)=O)cc4)oc23)ccn1] | 2-(4-(6-(4- (methylsulfonyl)phenyl)furo [3,2-d]pyrimidin-4- yl)pyridin-2-yl)propan-2-ol; |
| P1 | 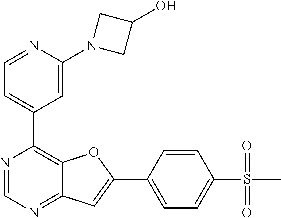 [SMILES: CS(=O)(=O)c1ccc(-c2cc3ncnc(-c4ccnc(N5CC(O)C5)c4)c3o2)cc1] | 1-(4-(6-(4- (methylsulfonyl)phenyl)furo [3,2-d]pyrimidin-4- yl)pyridin-2-yl)azetidin-3-ol |
| P2 | 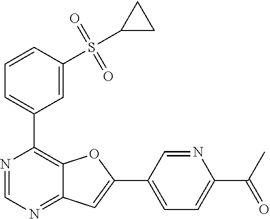 [SMILES: CC(=O)c1ccc(-c2cc3ncnc(-c4cccc(S(=O)(=O)C5CC5)c4)c3o2)cn1] | 1-(5-(4-(3- (cyclopropylsulfonyl)phenyl) furo[3,2-d]pyrimidin-6- yl)pyridin-2-yl)ethan-1-one |
| P3 | 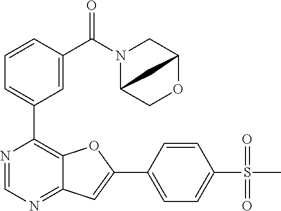 [SMILES: CS(=O)(=O)c1ccc(-c2cc3ncnc(-c4cccc(C(=O)N5C[C@H]6C[C@@H]5CO6)c4)c3o2)cc1] | ((1R,4R)-2-oxa-5- azabicyclo[2.2.1]heptan-5- yl)(3-(6-(4- (methylsulfonyl)phenyl)furo [3,2-d]pyrimidin-4- yl)phenyl)methanone |
| P4 | 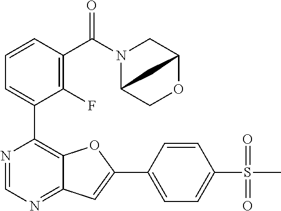 [SMILES: CS(=O)(=O)c1ccc(-c2cc3ncnc(-c4cccc(C(=O)N5C[C@H]6C[C@@H]5CO6)c4F)c3o2)cc1] | ((1R,4R)-2-oxa-5- azabicyclo[2.2.1]heptan-5- yl)(2-fluoro-3-(6-(4- (methylsulfonyl)phenyl)furo [3,2-d]pyrimidin-4- yl)phenyl)methanone |
| P5 | 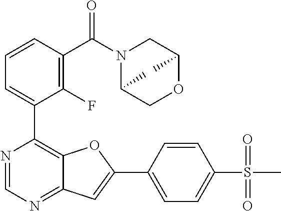 | ((1S,4S)-2-oxa-5- azabicyclo[2.2.1]heptan-5- yl)(2-fluoro-3-(6-(4- (methylsulfonyl)phenyl)furo [3,2-d]pyrimidin-4- yl)phenyl)methanone |
| P6 | 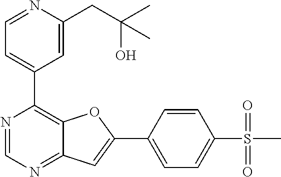 [SMILES: CC(C)(O)Cc1cc(-c2ncnc3cc(-c4ccc(S(C)(=O)=O)cc4)oc23)ccn1] | 2-methyl-1-(4-(6-(4- (methylsulfonyl)phenyl)furo [3,2-d]pyrimidin-4- yl)pyridin-2-yl)propan-2-ol |
| P7 | 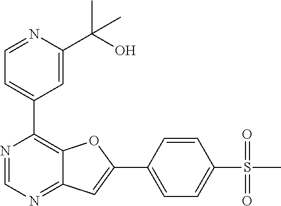 [SMILES: CC(C)(O)c1cc(-c2ncnc3cc(-c4ccc(S(C)(=O)=O)cc4)oc23)ccn1] | 2-(4-(6-(4- (methylsulfonyl)phenyl)furo [3,2-d]pyrimidin-4- yl)pyridin-2-yl)propan-2-ol |
| P8 | 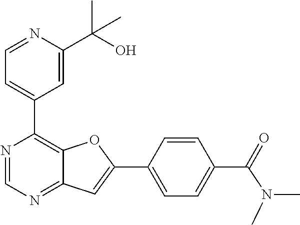 [SMILES: CN(C)C(=O)c1ccc(-c2cc3ncnc(-c4ccnc(C(C)(C)O)c4)c3o2)cc1] | 4-(4-(2-(2-hydroxypropan- 2-yl)pyridin-4-yl)furo[3,2- d]pyrimidin-6-yl)-N,N- dimethylbenzamide |
| P9 | 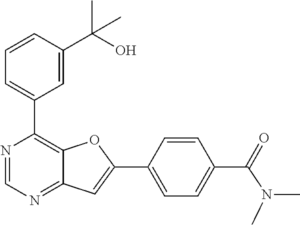 [SMILES: CN(C)C(=O)c1ccc(-c2cc3ncnc(-c4cccc(C(C)(C)O)c4)c3o2)cc1] | 4-(4-(3-(2-hydroxypropan- 2-yl)phenyl)furo[3,2- d]pyrimidin-6-yl)-N,N- dimethylbenzamide |
| P10 | 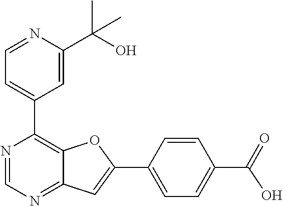 [SMILES: CC(C)(O)c1cc(-c2ncnc3cc(-c4ccc(C(=O)O)cc4)oc23)ccn1] | 4-(4-(2-(2-hydroxypropan- 2-yl)pyridin-4-yl)furo[3,2- d]pyrimidin-6-yl)benzoic acid |
| P11 | 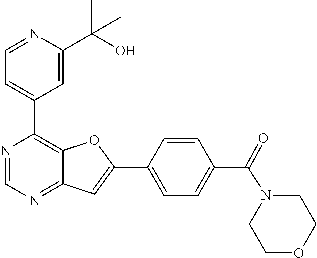 [SMILES: CC(C)(O)c1cc(-c2ncnc3cc(-c4ccc(C(=O)N5CCOCC5)cc4)oc23)ccn1] | (4-(4-(2-(2-hydroxypropan- 2-yl)pyridin-4-yl)furo[3,2- d]pyrimidin-6- yl)phenyl)(morpholino) methanone |
| P12 | 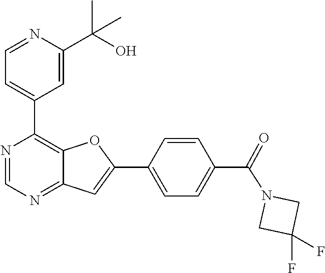 [SMILES: CC(C)(O)c1cc(-c2ncnc3cc(-c4ccc(C(=O)N5CC(F)(F)C5)cc4)oc23)ccn1] | (3,3-difluoroazetidin-1- yl)(4-(4-(2-(2- hydroxypropan-2- yl)pyridin-4-yl)furo[3,2- d]pyrimidin-6- yl)phenyl)methanone |
| P13 | 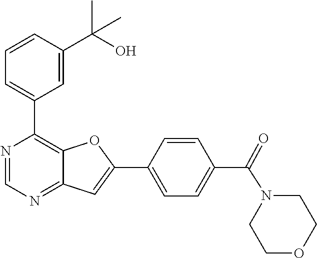 [SMILES: CC(C)(O)c1cccc(-c2ncnc3cc(-c4ccc(C(=O)N5CCOCC5)cc4)oc23)c1] | (4-(4-(3-(2-hydroxypropan- 2-yl)phenyl)furo[3,2- d]pyrimidin-6- yl)phenyl)(morpholino) methanone |
| P14 | 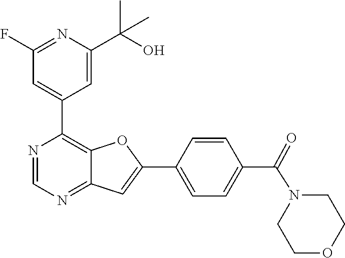 [SMILES: CC(C)(O)c1cc(-c2ncnc3cc(-c4ccc(C(=O)N5CCOCC5)cc4)oc23)cc(F)n1] | (4-(4-(2-fluoro-6-(2- hydroxypropan-2- yl)pyridin-4-yl)furo[3,2- d]pyrimidin-6- yl)phenyl)(morpholino) methanone |
| P15 | 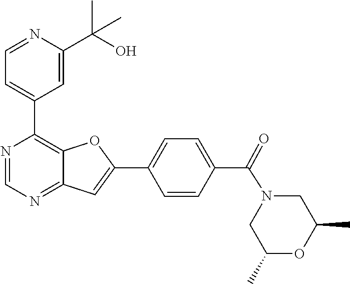 [SMILES: C[C@@H]1CN(C(=O)c2ccc(-c3cc4ncnc(-c5ccnc(C(C)(C)O)c5)c4o3)cc2)C[C@@H](C)O1] | ((2R,6R)-2,6- dimethylmorpholino)(4-(4- (2-(2-hydroxypropan-2- yl)pyridin-4-yl)furo[3,2- d]pyrimidin-6- yl)phenyl)methanone |
| P16 | 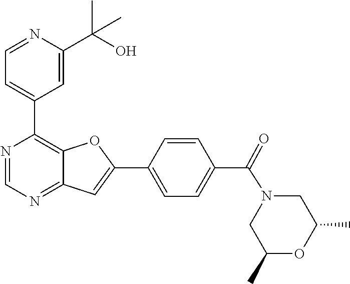 [SMILES: C[C@H]1CN(C(=O)c2ccc(-c3cc4ncnc(-c5ccnc(C(C)(C)O)c5)c4o3)cc2)C[C@H](C)O1] | ((2S,6S)-2,6- dimethylmorpholino)(4-(4- (2-(2-hydroxypropan-2- yl)pyridin-4-yl)furo[3,2- d]pyrimidin-6- yl)phenyl)methanone |
| P17 | 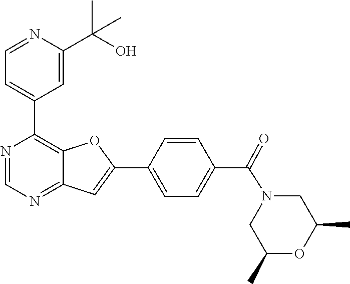 [SMILES: C[C@@H]1CN(C(=O)c2ccc(-c3cc4ncnc(-c5ccnc(C(C)(C)O)c5)c4o3)cc2)C[C@H](C)O1] | ((2S,6R)-2,6- dimethylmorpholino)(4-(4- (2-(2-hydroxypropan-2- yl)pyridin-4-yl)furo[3,2- d]pyrimidin-6- yl)phenyl)methanone |
| P18 | 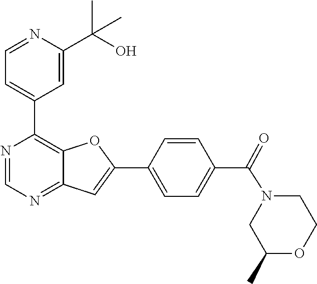 [SMILES: C[C@H]1CN(C(=O)c2ccc(-c3cc4ncnc(-c5ccnc(C(C)(C)O)c5)c4o3)cc2)CCO1] | (S)-(4-(4-(2-(2- hydroxypropan-2- yl)pyridin-4-yl)furo[3,2- d]pyrimidin-6-yl)phenyl)(2- methylmorpholino) methanone |
| P19 | 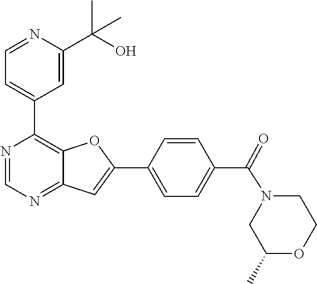 [SMILES: C[C@@H]1CN(C(=O)c2ccc(-c3cc4ncnc(-c5ccnc(C(C)(C)O)c5)c4o3)cc2)CCO1] | (R)-(4-(4-(2-(2- hydroxypropan-2- yl)pyridin-4-yl)furo[3,2- d]pyrimidin-6-yl)phenyl)(2- methylmorpholino) methanone |
| P20 | 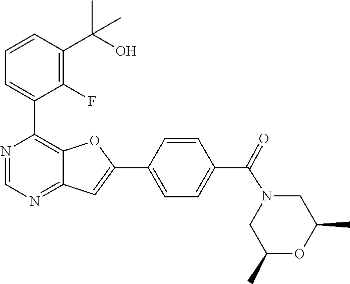 [SMILES: C[C@@H]1CN(C(=O)c2ccc(-c3cc4ncnc(-c5cccc(C(C)(C)O)c5F)c4o3)cc2)C[C@H](C)O1] | ((2S,6R)-2,6- dimethylmorpholino)(4-(4- (2-fluoro-3-(2- hydroxypropan-2- yl)phenyl)furo[3,2- d]pyrimidin-6- yl)phenyl)methanone |
| P21 | 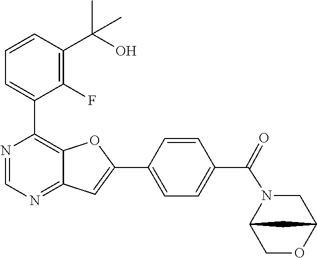 [SMILES: CC(C)(O)c1cccc(-c2ncnc3cc(-c4ccc(C(=O)N5C[C@H]6C[C@@H]5CO6)cc4)oc23)c1F] | ((1R,4R)-2-oxa-5- azabicyclo[2.2.1]heptan-5- yl)(4-(4-(2-fluoro-3-(2- hydroxypropan-2- yl)phenyl)furo[3,2- d]pyrimidin-6- yl)phenyl)methanone |
| P22 | 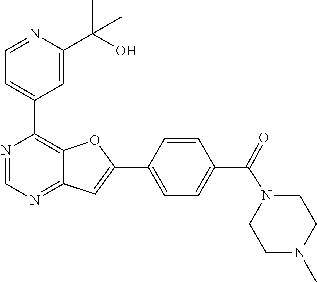 [SMILES: CN1CCN(C(=O)c2ccc(-c3cc4ncnc(-c5ccnc(C(C)(C)O)c5)c4o3)cc2)CC1] | (4-(4-(2-(2-hydroxypropan- 2-yl)pyridin-4-yl)furo[3,2- d]pyrimidin-6-yl)phenyl)(4- methylpiperazin-1- yl)methanone |
| P23 | 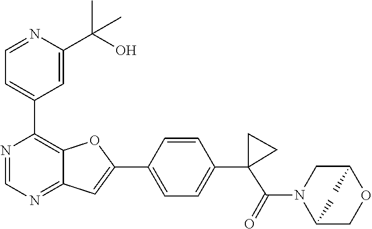 | ((1S,4S)-2-oxa-5- azabicyclo[2.2.1]heptan-5- yl)(1-(4-(4-(2-(2- hydroxypropan-2- yl)pyridin-4-yl)furo[3,2- d]pyrimidin-6- yl)phenyl)cyclopropyl) methanone |
| P24 | 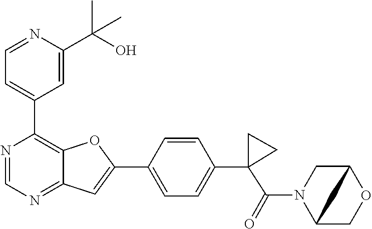 [SMILES: CC(C)(O)c1cc(-c2ncnc3cc(-c4ccc(C5(C(=O)N6C[C@H]7C[C@@H]6CO7)CC5)cc4)oc23)ccn1] | ((1R,4R)-2-oxa-5- azabicyclo[2.2.1]heptan-5- yl)(1-(4-(4-(2-(2- hydroxypropan-2- yl)pyridin-4-yl)furo[3,2- d]pyrimidin-6- yl)phenyl)cyclopropyl) methanone |
| P25 | 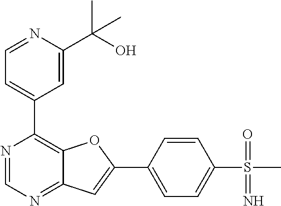 [SMILES: CC(C)(O)c1cc(-c2ncnc3cc(-c4ccc(S(C)(=N)=O)cc4)oc23)ccn1] | (4-(4-(2-(2-hydroxypropan- 2-yl)pyridin-4-yl)furo[3,2- d]pyrimidin-6- yl)phenyl)(imino)(methyl)- λ6-sulfanone |
| P26 | 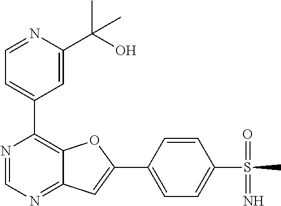 [SMILES: CC(C)(O)c1cc(-c2ncnc3cc(-c4ccc([S@@](C)(=N)=O)cc4)oc23)ccn1] | (S)-(4-(4-(2-(2- hydroxypropan-2- yl)pyridin-4-yl)furo[3,2- d]pyrimidin-6- yl)phenyl)(imino)(methyl)- λ6-sulfanone |
| P27 | 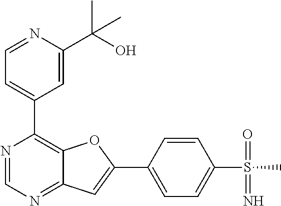 [SMILES: CC(C)(O)c1cc(-c2ncnc3cc(-c4ccc([S@](C)(=N)=O)cc4)oc23)ccn1] | (R)-(4-(4-(2-(2- hydroxypropan-2- yl)pyridin-4-yl)furo[3,2- d]pyrimidin-6- yl)phenyl)(imino)(methyl)- λ6-sulfanone |
| P28 | 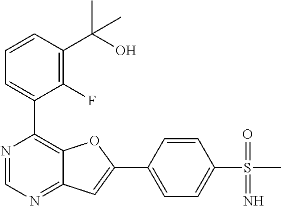 [SMILES: CC(C)(O)c1cccc(-c2ncnc3cc(-c4ccc(S(C)(=N)=O)cc4)oc23)c1F] | (4-(4-(2-fluoro-3-(2- hydroxypropan-2- yl)phenyl)furo[3,2- d]pyrimidin-6- yl)phenyl)(imino)(methyl)- λ6-sulfanone |
| P29 | 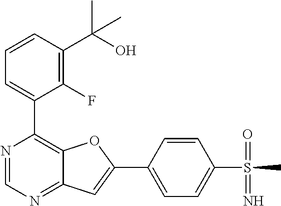 [SMILES: CC(C)(O)c1cccc(-c2ncnc3cc(-c4ccc([S@@](C)(=N)=O)cc4)oc23)c1F] | (S)-(4-(4-(2-fluoro-3-(2- hydroxypropan-2- yl)phenyl)furo[3,2- d]pyrimidin-6- yl)phenyl)(imino)(methyl)- λ6-sulfanone |
| P30 | 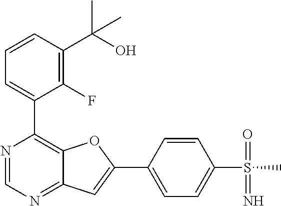 [SMILES: CC(C)(O)c1cccc(-c2ncnc3cc(-c4ccc([S@](C)(=N)=O)cc4)oc23)c1F] | (R)-(4-(4-(2-fluoro-3-(2- hydroxypropan-2- yl)phenyl)furo[3,2- d]pyrimidin-6- yl)phenyl)(imino)(methyl)- λ6-sulfanone |
| P31 | 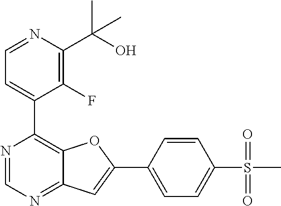 [SMILES: CC(C)(O)c1nccc(-c2ncnc3cc(-c4ccc(S(C)(=O)=O)cc4)oc23)c1F] | 2-(3-fluoro-4-(6-(4- (methylsulfonyl)phenyl)furo [3,2-d]pyrimidin-4- yl)pyridin-2-yl)propan-2-ol |
| P32 | 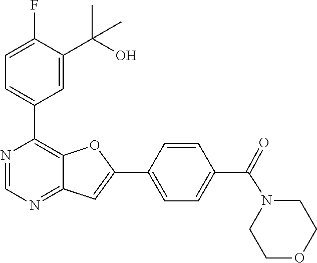 [SMILES: CC(C)(O)c1cc(-c2ncnc3cc(-c4ccc(C(=O)N5CCOCC5)cc4)oc23)ccc1F] | (4-(4-(4-fluoro-3-(2- hydroxypropan-2- yl)phenyl)furo[3,2- d]pyrimidin-6- yl)phenyl)(morpholino) methanone |
| P33 | 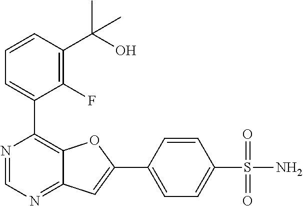 [SMILES: CC(C)(O)c1cccc(-c2ncnc3cc(-c4ccc(S(N)(=O)=O)cc4)oc23)c1F] | 4-(4-(2-fluoro-3-(2- hydroxypropan-2- yl)phenyl)furo[3,2- d]pyrimidin-6- yl)benzenesulfonamide |

and pharmaceutically acceptable solvates, salts, tautomers and stereoisomers thereof, including mixtures thereof in all ratios.

Surprisingly, in vitro studies show that small amounts of compounds according to the present invention are potent PI4K inhibitors. The invention therefore relates to compounds according to the present invention for the use for the inhibition of PI4K.

These versatile PI4K inhibitory properties make the compounds according to the present invention ideal candidates for treatment and/or prevention of PI4K-related disorders such as, but not limited to, protozoan infections and viral infections.

Surprisingly, small amounts of the compounds according to the present invention are sufficient to decrease Plasmodium viability and reduce growth. Additional data suggests a high inhibitory potential of said compounds on Plasmodium PI4K activity. The present invention, therefore, also relates to the use of the compounds according to the invention for treatment and/or prevention of protozoan infections such as malaria.

The compounds according to the present invention inhibit PI4K of protozoans such as, but not limited to, Plasmodium ssp, Toxoplasma ssp, Babesia ssp, Cryptosporidium ssp.

Human PI4K is a well-known druggable-target for the treatment and prevention of virus infections. Surprisingly, the disclosed compounds were additionally found to inhibit human PI4KIIIβ, an important target for viruses such as but not limited to RNA viruses.

Therefore, some embodiments of the present invention relate to the use of the compounds according to the invention for treatment and/or prevention of PI4K-related disorder selected from the list of protozoan infections and viral infections. In a preferred embodiment, said PI4K-related disorder is a protozoan infection, more preferably malaria.

Viral infections can be caused by viruses such as RNA or DNA viruses. In a preferred embodiment, the compounds according to the present invention are used for the treatment and/or prevention of virus infections caused by RNA viruses.

In a preferred embodiment said virus infection is caused by viruses selected from the families orthomyxoviridae, adenoviridae, paramyxoviridae, and coronaviridae. Virus of the orthomyxoviridae family include the influenza A virus, influenza B virus, influenza C virus, the infectious salmon anemia virus (isavirus), Thogoto Virus, and Dhori Virus. Members of the adenoviridae family include human adenovirus A, B, C, D, E, and F; bovine adenovirus A, B, and C; canine adenovirus; equine adenovirus A and B; murine adenovirus A; ovine adenovirus A and B; porcine adenovirus A, B, and C; and tree shrew adenovirus. Members of the paramyxoviridae family include bovine parainfluenza virus 3 (BPIV-3), human parainfluenza virus 1 (HPIV-1), human parainfluenza virus 3 (HPIV-3); sendai virus (murine parainfluenza virus 1); simian parainfluenza virus 10 (SPIV-10), bovine respiratory syncytial virus (BRSV), human respiratory syncytial virus (HRSV), pneumonia virus of mice (PVM), canine distemper virus (CDV), dolphin distemper virus (DMV), measles virus (MeV), Peste des petits ruminants virus (PPRV), phocine (seal) distemper virus (PDV), porpoise distemper virus, rinderpest virus (RPV), avian paramyxovirus 2 (APMV-2), avian paramyxovirus 3 (APMV-3), avian paramyxovirus 4 (APMV-4), avian paramyxovirus 5 (APMV-5), avian paramyxovirus 6 (APMV-6), avian paramyxovirus 7 (APMV-7), avian paramyxovirus 8 (APMV-8), avian paramyxovirus 9 (APMV-9), human parainfluenza virus 2 (HPIV-2), human parainfluenza virus 4a (HPIV-4a), human parainfluenza virus 4b (HPIV-4-b), mumps virus, newcastle disease virus (avian paramyxovirus 1) (NDV; APMV-1), porcine rubulavirus, simian parainfluenza virus 5 (SV-5), and simian parainfluenza virus 41 (SV-41). Members of the coronaviridae family include infectious bronchitis virus, bovine coronavirus, canine coronviarus, feline coronavirus, human coronavirus, and SARS-coronavirus, SAR2-Coronavirus-2, MERS-CoV. In a more preferred embodiment, the compounds according to the present invention are used for treatment and/or prevention of SARS-CoV2.

While the compounds according to the present invention were able to inhibit both protozoan and human PI4K, surprisingly protozoan PI4K was preferentially inhibited. Human PI4K did show inhibition upon treatment with the compounds according to the present invention. However, since protozoan PI4K was inhibited at very low concentration, the compounds according to the present invention are especially useful for the treatment of protozoan infection using low amounts without or with low cross inhibition of human PI4K. These properties have a substantial pharmacological potential as inhibition of human PI4K can result in significant side effects in some patients. The excellent inhibitory properties of the compounds according to the present invention allow for treatment and/or prevention of PI4K-related diseases using very low amounts, thereby lowering the likelihood of toxicological effects caused by the compounds and allowing lower concentrations of additional established antimalarials useful in combination therapy.

### Compositions

The present invention further relates to a pharmaceutical composition comprising at least one compound according to formula (I) of the present invention.

In another particular embodiment, a pharmaceutical formulation is provided containing at least one compound according to formula (I) of the present invention and a pharmaceutically acceptable carrier, diluent or excipient thereof.

The present invention further relates to said pharmaceutical compositions for use in prevention and/or treatment of PI4K-related disorders.

In some embodiments the present invention further relates to pharmaceutical compositions for use in prevention and/or treatment of PI4K-related disorders, comprising at least one compound of formula (I) according to the present invention, wherein the PI4K-related disorders are selected from the list of protozoan infections and viral infections. In a preferred embodiment, said PI4K-related disorder is caused by an RNA virus. In another preferred embodiment said PI4K-related disorder is malaria.

### Combination

According to the present invention, a compound according to formula (I) or a pharmaceutical composition thereof can be administered alone or in combination with a further active ingredient (a co-agent) such as a pharmaceutically active compound useful in the treatment and/or prevention of PI4K-related disorders.

Therefore, the present invention further also refers to a pharmaceutical composition comprising at least one compound of formula (I) and at least one further active ingredient (co-agent), which is different from formula (I). In certain embodiments said co-agent is an antimalarial agent, which is different from formula (I). Preferably, said further active ingredient (the antimalarial co-agent) is selected from: Pyronaridine (free base or tetraphosphate salt), quinacrine, chloroquine, ferroquine, primaquine, tafenoquine, doxycycline, atovaquone, proguanil, cycloguanil, cabamiquine (free base or succinate salt), cipargamin, ganaplacide, sulfadoxine, pyrimethamine, artemisinin, dihydrodroartemisinin, artesunic acid, artesunate, arterolane, artefenomel, lumefantrine, DSM 265 (CAS Number: 1282041-94-4), (OC-6-21)-[4-[[2-(1,1-Difluoroethyl)-5-methyl[1,2,4]triazolo[1,5-a]pyrimidin-7-yl]amino]phenyl]pentafluorosulfur, SAR121 (CAS Number: 2260904-47-8), Benzamide, 5-[2-[3-[[(aminoiminomethyl)amino]carbonyl]-5-(trifluoromethyl)phenyl]ethynyl]-N-2-pyridinyl-2-(trifluoromethyl), INE963 (CAS number 2640567-43-5), 4-Piperidinol, 4-(aminomethyl)-1-[5-[2-methoxy-6-(1-methylethyl)-3-pyridinyl]imidazo[2,1-b]-1,3,4-thiadiazol-2-yl], ZY19489 (CAS Number: 1821293-40-6), 2,4-Pyrimidinediamine, N2-(4-cyclopropyl-5-fluoro-6-methyl-2-pyridinyl)-5-[(3R)-3,4-dimethyl-1-piperazinyl]-N4-(1,5-dimethyl-1H-pyrazol-3-yl) and GSK701 (Cas Number: 2366983-10-8) Methanone, [(3R)-3-(4-fluorophenyl)-1-pyrrolidinyl.

In another embodiments the pharmaceutical composition comprises at least one compound of formula (I) and at least one additional antiviral agent (a antiviral co-agent), which is different from formula (I).

An antiviral co-agent according to the present invention can be any antiviral agent known in the art such as but not limited to antivirals selected from the list of: Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost, Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Ensitrelvir, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvada, Umifenovir, Valaciclovir (Valtrex), Valganciclovir (Valcyte), Vicriviroc, Vidarabine Zalcitabine, Zanamivir (Relenza), Zidovudine.

### Administration

The invention encompasses the administration of compounds according to the invention or of a pharmaceutical formulation (=pharmaceutical preparation, pharmaceutical composition) thereof, wherein said compounds or the pharmaceutical formulation thereof is administered to an individual prior to, simultaneously or sequentially with other therapeutic regimens or co-agents useful in the treatment of PI4K-related diseases such as malaria or viral infections (e.g., multiple drug regimens), in an effective amount. Compounds according to the present invention or the pharmaceutical formulations thereof that are administered simultaneously with said co-agents can be administered in the same or different composition(s) and by the same or different route(s) of administration.

In further embodiments, the present invention relates to a method for preventing or treating of PI4K-related disorders, wherein the method comprises the following step:
(i) providing at least one compound and/or a pharmaceutical composition according to the present invention; and
(ii) administering an effective amount of said at least one compound or said composition to a patient in need thereof.

In a preferred embodiment said PI4K-related disorder is selected from the list of protozoan infections and viral infection, more preferably a viral infection caused by an RNA virus and most preferably malaria.

Further preferred embodiments listed above also apply to the method according to the present invention.

The invention furthermore relates to medicaments comprising at least one compound of formula (I) and/or pharmaceutically acceptable derivatives, solvates and stereoisomers thereof, including mixtures thereof in all ratios, and optionally excipients and/or adjuvants for the treatment and/or prevention of PI4K-related disorders.

Pharmaceutical compositions can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical compositions can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

In some embodiments, the administration according to the method of the present invention takes place oral, including buccal or sublingual, rectal, nasal, topical, including buccal, sublingual or transdermal, vaginal or parenteral, including subcutaneous, intramuscular, intravenous or intradermal.

Pharmaceutical compositions adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatin shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubilize, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatin, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatin or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acacia mucilage or solutions of cellulose or polymer materials and pressing it through a sieve. As an alternative to granulation, the powder mixture can be run through a tableting machine, giving lumps of non-uniform shape, which are broken up to form granules. The granules can be lubricated by addition of stearic acid, a stearate salt, talc or mineral oil in order to prevent sticking to the tablet casting molds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be pre, pared in the form of dosage units so that a given quantity comprises a pre-specified amount of the compound. Syrups can be prepared by dissolving the compound in an aqueous solution with a suitable flavor, while elixirs are prepared using a non-toxic alcoholic vehicle. Suspensions can be formulated by dispersion of the compound in a non-toxic vehicle. Solubilizers and emulsifiers, such as, for example, ethoxylated isostearyl alcohols and polyoxyethylene sorbitol ethers, preservatives, flavor additives, such as, for example, peppermint oil or natural sweeteners or saccharin, or other artificial sweeteners and the like, can likewise be added. The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of formula (I) and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula (I) and the salts, solvates and physiologically functional derivatives thereof can also be delivered using a delivery reagent such as monoclonal antibodies, nucleic acids or nanoparticles as individual carriers to which the compound molecules are coupled or enclosed. The compounds can also be coupled to soluble polymers as targeted medicament carriers. Such polymers may encompass polyvinylpyrrolidone, pyran copolymer, polyhydroxypropylmethacrylamidophenol, polyhydroxy-ethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-capro-lactone, polyhydroxybutyric acid, polyorthoesters, polyacetals, polydihy-droxypyrans, polycyanoacrylates and crosslinked or amphipathic block co-polymers of hydrogels.

Pharmaceutical compositions adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986). Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical compositions adapted for topical application to the eye include eye drops, in which the active ingredient is dissolved or suspended in a suitable carrier, in particular an aqueous solvent.

Pharmaceutical compositions adapted for topical application in the mouth encompass lozenges, pastilles and mouthwashes.

Pharmaceutical compositions adapted for rectal administration can be administered in the form of suppositories or enemas.

Pharmaceutical compositions adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil. Pharmaceutical formulations adapted for administration by inhalation encompass finely particulate dusts or mists, which can be generated by various types of pressurized dispensers with aerosols, nebulizers or insufflators.

Pharmaceutical compositions adapted for vaginal administration can be administered as pessaries, tampons, creams, gels, pastes, foams or spray formulations.

Pharmaceutical compositions adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the compositions may also comprise other agents usual in the art with respect to the particular type of formulation; thus, for example, formulations which are suitable for oral administration may comprise flavors.

A therapeutically effective amount of a compound of the formula (I) depends on a number of factors, including, for example, the age and weight of the subjects such as animals and humans, the precise condition that requires treatment, and its severity, the nature of the formulation and the method of administration, and is ultimately determined by the treating doctor or vet. However, an effective amount of a compound according to the invention is generally in the range from 0.01 to 100 mg/kg of body weight of the recipient (mammal) per day and particularly typically in the range from 1 to 100 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention perse. It can be assumed that similar doses are suitable for the treatment of other conditions mentioned above.

### Preparation of Reactants and Compounds According to the Invention

Compounds and derivatives thereof according to the present invention can be prepared from readily available starting materials using methods and procedures known to the skilled person. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures.

A general synthetic approach for obtaining compounds of formula (I) is depicted in Scheme 1 below.

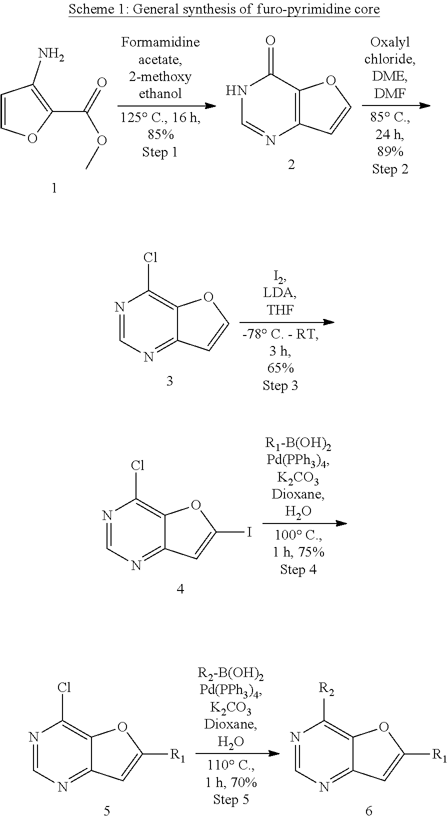

If the above synthetic methods are not applicable to obtain furo-pyridine or furo-pyrimidine derivatives and/or necessary intermediates according to the invention, suitable methods of preparation known by a person skilled in the art should be used. In general, the synthesis pathways for any individual furo-pyridine or furo-pyrimidine derivative will depend on the specific substituents of each molecule and upon the ready availability of intermediates necessary, again, such factors being appreciated by those of ordinary skill in the art.

Further examples illustrating different synthesis strategies to obtain compounds or reactants according to the present invention can be found in the examples disclosed below.

### EXAMPLES

### HPLC:

LC purity traces were performed using one of the following methods:

### Method 1:

Using a Kinetex 2.6 μM C-18 column, 2 μL injection volume, flow 0.7 mL/min; gradient: 15-100% B in 1.2 min (hold 3.3 min), 100-15% in 0.3 min (hold 1.2 min) (Mobile phase A: 10 mM buffer (Ammonium acetate/acetic acid) in H₂O and Mobile phase B: 10 mM buffer (Ammonium acetate/acetic acid) in Methanol).

### Method 2:

Using a Kinetex 1.7 μM C-18 column, 1 μL injection volume, flow 1.2 mL/min; gradient: 5-100% B in 1.5 min (hold 0.4 min), 100-5% in 0.3 min (hold 0.5 min) (Mobile phase A: 0.1% formic acid in H2O and Mobile phase B: 0.1% formic acid in Acetonitrile).

The invention will be illustrated, but not limited, by reference to the specific embodiments described in the following examples.

Unless otherwise specified, all starting materials are obtained from commercial suppliers and used without further purifications. Unless otherwise specified, all temperatures are expressed in ° C. and all reactions are conducted at rt.

Compounds were purified by either silica chromatography or preparative HPLC.

Unless stated otherwise all structures indicated below, where no specific stereochemistry is indicated, refer to mixtures of the stereoisomers (preferably a racemic mixture of the stereoisomers).

### Example 1: Synthesis of Furo-Pyrimidine Core

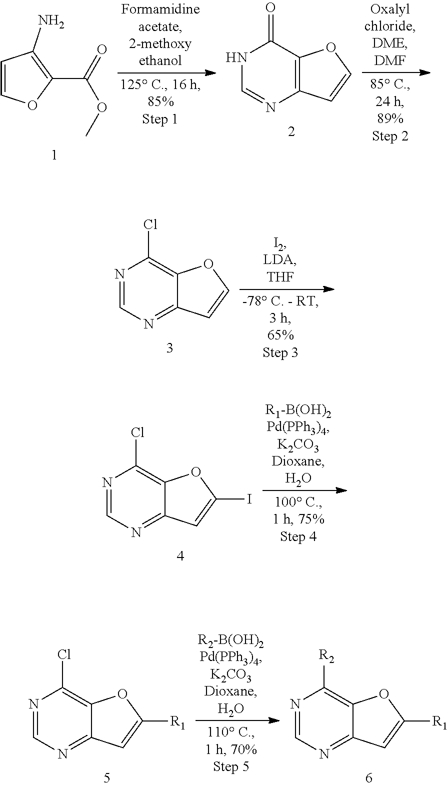

Step-1: A mixture of methyl 3-aminothiophene-2-carboxylate (5 g, 0.035 mol), formamidine acetate (7.3 g, 0.070 g) and 2-methoxy ethanol (50 mL) was stirred and heated to reflux for 3 hours. The mixture was cooled to ambient temperature and water (50 ml) was added. The resultant solid was isolated, washed thoroughly with water and with diethyl ether and dried under vacuum to get furo[3,2-d]pyrimidin-4(3H)-one 2 (4.1 g, 85.06%). LCMS: Calculated for C6H6N6O2 136.11, Observed 137.1 (M+H), RT. 0.934 min, 94.28% (Max). 1H NMR (400 MHz, DMSO-d6): δ 12.60 (s, 1H), 8.23 (d, J=1.60 Hz, 1H), 8.07 (s, 1H), 6.99 (d, J=2.00 Hz, 1H).

Step-2: Furo[3,2-d]pyrimidin-4(3H)-one 2 (4.1 g) was taken in thionylchloride (20 ml) and DMF (0.2 ml). Stirred at reflux for 5 h. After the completion of the reaction, the reaction mixture was concentrated to remove thionylchloride, was added water, extracted using dichloromethane and dried with anhydrous sodium sulphate. After concentrated under vacuum, trituration was carried out with hexane to get 4-chlorofuro[3,2-d]pyrimidine 3 (4.1 g, 89.1%). LCMS: Calculated for C6H3ClN2O 154.55, Observed 155.1 (M+H), RT. 1.41 min, 97.42% (Max). 1H NMR (400 MHz, DMSO-d6): δ 8.92 (s, 1H), 8.68 (d, J=2.40 Hz, 1H), 7.40 (d, J=2.00 Hz, 1H).

Step-3: To a stirred solution of 4-chlorofuro[3,2-d]pyrimidine 3 (2 g, 0.013 mol) in THF at −78° C., was added dropwise n-butyl lithium 1.6 M solution in THF (12.1 ml, 0.019 mol) over a period of 15 minutes. After 2 h stirring at −78° C., was added ICI (1.01 ml, 0.019 mol) dropwise and allowed to stir at room temperature for 30 minutes. The reaction mixture is poured into water and extracted three times with ethyl acetate. The combined organic phases are washed with 10% sodium thiosulphate solution, dried over sodium sulphate and after filtration, dried under reduced pressure. The solid obtained was washed with diethyl ether and dried to get 4-chloro-6-iodofuro[3,2-d]pyrimidine 4 (2 g, 55.2%) as reddish orange solid. LCMS: Calculated for C6H2ClIN2O 280.45, Observed 280.9 (M+H), RT. 1.94 min, 95.31% (Max). 1H NMR (400 MHz, DMSO-d6): δ 8.85 (s, 1H), 7.72 (s, 1H).

### General Procedure for Step 4:

To a stirred solution of 4-chloro-6-iodofuro[3,2-d]pyrimidine (0.285 mmol), R1-B(OH)2 (67.0 mg, 0.285 mmol) in 1,4-Dioxane (3 ml) and Water (1.00 ml) and added K2CO3 (0.571 mmol). The reaction mixture was then degassed for 5 min followed by addition of Pd(PPh3)4 (0.029 mmol). The reaction mixture was stirred for about 3 h at 90° C. The solvent was evaporated to get compound 5 as crude mixture which was taken as is for next step.

### MANUFACTURING EXAMPLES

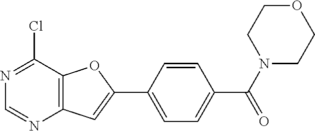 [SMILES: O=C(c1ccc(-c2cc3ncnc(Cl)c3o2)cc1)N1CCOCC1]

4-(4-{4-chlorofuro[3,2-d]pyrimidin-6-yl}benzoyl)morpholine (building block): LCMS: Calculated 343.77; Observed 344.0(M+H).

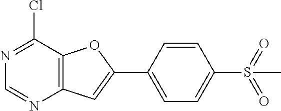 [SMILES: CS(=O)(=O)c1ccc(-c2cc3ncnc(Cl)c3o2)cc1]

4-chloro-6-(4-methanesulfonylphenyl)furo[3,2-d]pyrimidine (building block): LCMS: Calculated for C13H9ClN2O3S, Exact Mass: 308.73, Observed 309.1(M+H).

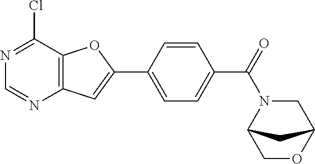 [SMILES: O=C(c1ccc(-c2cc3ncnc(Cl)c3o2)cc1)N1C[C@H]2C[C@@H]1CO2]

(1R,4R)-5-(4-{4-chlorofuro[3,2-d]pyrimidin-6-yl}benzoyl)-2-oxa-5-azabicyclo[2.2.1]heptane (building block): LCMS: Calculated for C18H14ClN3O3, Exact mass 355.07, Observed 356.0 (M+H).

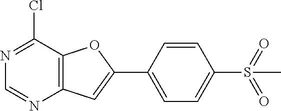 [SMILES: CS(=O)(=O)c1ccc(-c2cc3ncnc(Cl)c3o2)cc1]

4-chloro-6-(4-methanesulfonylphenyl)furo[3,2-d]pyrimidine (building block): LCMS: Calculated for C21H18FN3O4S 308.0, Observed 309.0 (M+H).

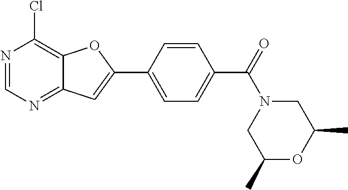 [SMILES: C[C@@H]1CN(C(=O)c2ccc(-c3cc4ncnc(Cl)c4o3)cc2)C[C@H](C)O1]

(2R,6S)-4-(4-{4-chlorofuro[3,2-d]pyrimidin-6-yl}benzoyl)-2,6-dimethylmorpholine (building block): LCMS: Calculated for C19H18ClN3O3 371.82, 372.0 (M+H).

### General Procedure for Step 5:

To a stirred solution of compound 5 (0.233 mmol), R2-B(OH)2 (0.233 mmol) in 1,4-Dioxane (4 ml) and Water (1 ml) added potassium carbonate (0.465 mmol). The reaction mixture was then degassed for 5 min followed by addition of Pd(PPh3)4 (0.029 mmol) and stirred for about 16 h at 110° C. The resulting residue was purified by preparative-HPLC to get compound 6.

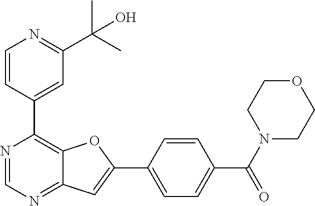 [SMILES: CC(C)(O)c1cc(-c2ncnc3cc(-c4ccc(C(=O)N5CCOCC5)cc4)oc23)ccn1]

2-(4-{6-[4-(morpholine-4-carbonyl)phenyl]furo[3,2-d]pyrimidin-4-yl}pyridin-2-yl)propan-2-ol: 1H NMR (400 MHz, DMSO-d6): δ 8.38 (s, 1H), 8.20 (s, 1H), 8.03 (d, J=5.60 Hz, 1H), 7.72-7.70 (m, 1H), 7.50 (d, J=8.40 Hz, 2H), 6.90 (s, 3H), 3.01-2.52 (m, 8H), 0.90 (s, 6H). LCMS: Calculated for C25H24N4O4 343.77, Observed 444.49(M+H).

### MANUFACTURING EXAMPLES

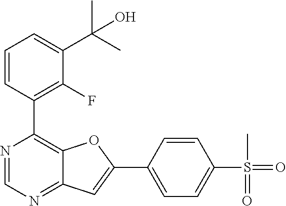 [SMILES: CC(C)(O)c1cccc(-c2ncnc3cc(-c4ccc(S(C)(=O)=O)cc4)oc23)c1F]

2-{2-fluoro-3-[6-(4-methanesulfonylphenyl)furo[3,2-d]pyrimidin-4-yl]phenyl}propan-2-ol: 1H NMR (400 MHz, DMSO-d6): δ 9.20 (s, 1H), 8.28 (d, J=8.40 Hz, 2H), 8.15 (d, J=8.80 Hz, 2H), 8.10 (s, 1H), 7.95-7.91 (m, 1H), 7.84-7.80 (m, 1H), 7.46 (t, J=7.60 Hz, 1H), 5.50 (s, 1H), 3.31 (s, 3H), 1.62 (s, 6H). LCMS: Calculated for C22H19FN2O4S, Molecular Weight: 426.1, Observed 427.1 (M+H).

(4-(4-(2-fluoro-3-(2-hydroxypropan-2-yl)phenyl)furo[3,2-d]pyrimidin-6-yl)phenyl)(imino)(methyl)-λ⁶-sulfanone (D203): 1H NMR (400 MHz, DMSO): δ 9.18 (s, 1H), 8.23-8.21 (d, J=8.4 Hz, 2H), 8.12-8.10 (d, J=8.4 Hz, 2H), 8.045 (s, 1H), 7.94-7.91 (m, 1H), 7.83-7.80 (t, J=6.8 Hz, 1H), 7.47-7.43 (t, J=7.8 Hz, 1H), 5.48 (s, 1H), 4.39 (s, 1H), 3.14 (s, 3H), 1.61 (s, 6H). LCMS: Calculated for C22H20FN3O3S, 425.478, Observed 426.2 (M+H).

Separation of the (S)- and (R)-enantiomer (D209 and D210 respectively) was performed using the following method with a Waters 2545 Quaternary gradient Module with MassLynx Software (Version 4.1), Waters 2424 ELS Detector, Waters 2767 Sample Manager and a Chiralpak IC 5 μM, (20 mm×250 mm) Chiral Column. Isocratic Elution: Hexane/CH₂Cl₂/EtOH (50:25:25) D209 t_{R}=20.18 min (purity 100%), D210 t_{R}=29.02 min (purity 100%).

(S)-(4-(4-(2-fluoro-3-(2-hydroxypropan-2-yl)phenyl)furo[3,2-d]pyrimidin-6-yl)phenyl)(imino)(methyl)-λ⁶-sulfanone (D209). Synthesis: See D203 above. Yield 43%. 1H NMR (300 MHz, MeOD-d₄) δ=9.11 (s, 1H), 8.19 (q, J=8.4 Hz, 4H), 7.95 (t, J=7.8 Hz, 1H), 7.80 (d, J=7.1 Hz, 1H), 7.71 (s, 1H), 7.44 (t, J=7.7 Hz, 1H), 3.23 (s, 3H), 1.72 (s, 6H); Anal. RP-HPLC t_{R}=2.34 min (method 2, purity 100%); LC-MS: m/z=426.2 [M+H]⁺ (anal. calcd for C₂₂H₂₀FN3O3S⁺: m/z=426.1).

(R)-(4-(4-(2-fluoro-3-(2-hydroxypropan-2-yl)phenyl)furo[3,2-d]pyrimidin-6-yl)phenyl)(imino)(methyl)-λ⁶-sulfanone (D210). Synthesis: See D203 above. Yield 46%. ¹H NMR (300 MHz, DMSO-d₆) δ=9.25 (s, 1H), 8.29 (overlapping s, 2H), 8.22-8.09 (m, 3H), 7.99 (t, J=7.8 Hz, 1H), 7.88 (t, J=6.7 Hz, 1H), 7.52 (t, J=7.7 Hz, 1H), 5.59 (s, 1H), 3.20 (s, 3H), 1.68 (s, 6H); Anal. RP-HPLC t_{R}=2.34 min (method 2, purity 99%); LC-MS: m/z=426.2 [M+H]⁺ (anal. calcd for C₂₂H₂₀FN₃O₃S⁺: m/z=426.1).

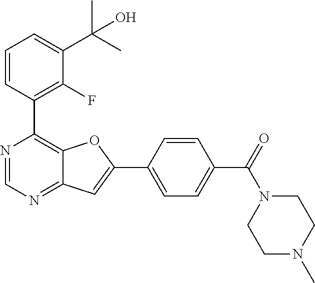 [SMILES: CN1CCN(C(=O)c2ccc(-c3cc4ncnc(-c5cccc(C(C)(C)O)c5F)c4o3)cc2)CC1]

2-(2-fluoro-3-{6-[4-(4-methylpiperazine-1-carbonyl)phenyl]furo[3,2-d]pyrimidin-4-yl}phenyl)propan-2-ol: 1H NMR (400 MHz, DMSO-d6): δ 9.81 (s, 1H), 9.18 (s, 1H), 8.12 (d, J=8.00 Hz, 2H), 7.98 (s, 1H), 7.93-7.80 (m, 1H), 7.67 (d, J=8.00 Hz, 2H), 7.45 (t, J=8.00 Hz, 1H), 3.13 (s, 4H), 2.84 (s, 4H), 1.62 (s, 6H). LCMS: Calculated for C26H27FN4O3 474.5, Observed 475.2 (M+H).

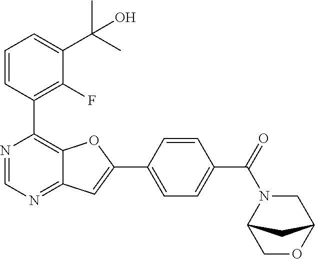 [SMILES: CC(C)(O)c1cccc(-c2ncnc3cc(-c4ccc(C(=O)N5C[C@H]6C[C@@H]5CO6)cc4)oc23)c1F]

2-[2-fluoro-3-(6-{4-[(1R,4R)-2-oxa-5-azabicyclo[2.2.1]heptane-5-carbonyl]phenyl}furo[3,2-d]pyrimidin-4-yl)phenyl]propan-2-ol: 1H NMR (400 MHz, DMSO-d6): δ 9.14 (s, 1H), 8.08 (d, J=8.40 Hz, 2H), 7.95-7.91 (m, 1H), 7.81-7.78 (m, 2H), 7.71 (d, J=8.00 Hz, 2H), 7.43 (t, J=7.60 Hz, 1H), 5.21 (s, 1H), 4.63 (s, 2H), 3.88 (d, J=7.20 Hz, 1H), 3.75 (s, 1H), 3.54 (dd, J=1.20, 11.00 Hz, 1H), 3.35 (d, J=9.60 Hz, 1H), 1.92 (d, J=8.80 Hz, 1H), 1.83 (s, 1H), 1.64 (s, 6H). LCMS: Calculated for C27H24FN3O4, Exact mass 473.18, Observed 474.2 (M+H).

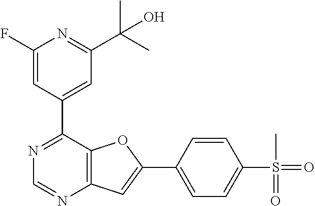 [SMILES: CC(C)(O)c1cc(-c2ncnc3cc(-c4ccc(S(C)(=O)=O)cc4)oc23)cc(F)n1]

2-{6-fluoro-4-[6-(4-methanesulfonylphenyl)furo[3,2-d]pyrimidin-4-yl]pyridin-2-yl}propan-2-ol: 1H NMR (400 MHz, DMSO-d6): δ 9.27 (s, 1H), 8.78 (s, 1H), 8.45 (d, J=8.00 Hz, 2H), 8.19 (d, J=7.20 Hz, 3H), 7.97 (s, 1H), 7.65-7.56 (m, 1H), 5.69 (s, 1H), 3.44 (s, 3H), 1.53 (s, 6H). LCMS: Calculated for C21H18FN3O4S 427.45, Observed 428.0 (M+H).

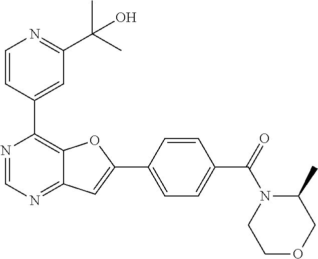 [SMILES: C[C@H]1COCCN1C(=O)c1ccc(-c2cc3ncnc(-c4ccnc(C(C)(C)O)c4)c3o2)cc1]

2-[4-(6-{4-[(3S)-3-methylmorpholine-4-carbonyl]phenyl}furo[3,2-d]pyrimidin-4-yl)pyridin-2-yl]propan-2-ol: 1H NMR (400 MHz, DMSO-d6): δ 9.23 (s, 1H), 8.85-8.83 (m, 1H), 8.56-8.56 (m, 1H), 8.35-8.33 (m, 1H), 8.08-8.06 (m, 1H), 7.63-7.61 (m, 3H), 7.38 (s, 1H), 4.88 (s, 1H), 3.95 (s, 1H), 3.72-3.69 (m, 2H), 3.58-3.45 (m, 2H), 1.72 (s, 6H), 1.44 (d, J=6.80 Hz, 3H). LCMS: Calculated for C26H26N4O4 458.52, Observed 459.1 (M+H).

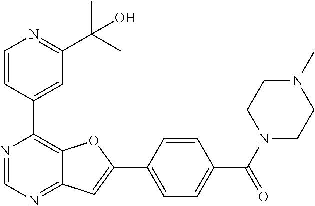 [SMILES: CN1CCN(C(=O)c2ccc(-c3cc4ncnc(-c5ccnc(C(C)(C)O)c5)c4o3)cc2)CC1]

2-(4-{6-[4-(4-methylpiperazine-1-carbonyl)phenyl]furo[3,2-d]pyrimidin-4-yl}pyridin-2-yl)propan-2-ol: 1H NMR (400 MHz, DMSO-d6): δ 9.20 (s, 1H), 8.84-8.83 (m, 2H), 8.32-8.26 (m, 3H), 7.99 (s, 1H), 7.64 (d, J=8.40 Hz, 2H), 5.50 (s, 1H), 3.66 (s, 2H), 3.32-3.36 (m, 2H), 2.38 (S, 3H), 2.33-2.34 (m, 1H), 2.22 (s, 3H), 1.55 (s, 6H). LCMS: Calculated for C26H27N5O3 457.53, Observed 458.1 (M+H).

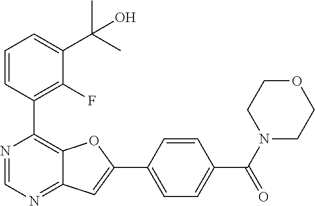 [SMILES: CC(C)(O)c1cccc(-c2ncnc3cc(-c4ccc(C(=O)N5CCOCC5)cc4)oc23)c1F]

2-(2-fluoro-3-{6-[4-(morpholine-4-carbonyl)phenyl]furo[3,2-d]pyrimidin-4-yl}phenyl)propan-2-ol: 1H NMR (400 MHz, DMSO-d6): δ 9.16 (s, 1H), 8.09 (d, J=8.40 Hz, 2H), 7.93 (d, J=6.00 Hz, 2H), 7.81 (s, 1H), 7.63 (d, J=8.00 Hz, 2H), 7.45 (t, J=8.0 Hz, 1H), 5.50 (s, 1H), 3.65 (s, 8H), 1.61 (s, 6H). LCMS: Calculated for C26H24FN3O4 461.493, Observed 462.1 (M+H).

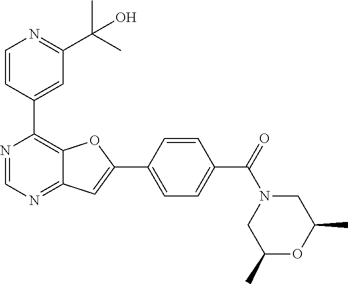 [SMILES: C[C@@H]1CN(C(=O)c2ccc(-c3cc4ncnc(-c5ccnc(C(C)(C)O)c5)c4o3)cc2)C[C@H](C)O1]

2-[4-(6-{4-[(2R,6S)-2,6-dimethylmorpholine-4-carbonyl]phenyl}furo[3,2-d]pyrimidin-4-yl)pyridin-2-yl]propan-2-ol: 1H NMR (400 MHz, DMSO-d6): δ 9.21 (s, 1H), 8.84-8.82 (m, 2H), 8.33-8.27 (m, 3H), 7.99 (s, 1H), 7.66 (d, J=8.40 Hz, 2H), 5.50 (s, 1H), 4.43-4.40 (m, 1H), 3.59-3.50 (m, 2H), 3.44-3.27 (m, 1H), 2.68-2.56 (m, 1H), 1.55 (s, 6H), 1.40-1.10 (m, 6H). LCMS: Calculated for C27H28N4O4 472.55, Observed 473.3 (M+H).

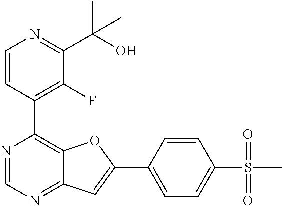 [SMILES: CC(C)(O)c1nccc(-c2ncnc3cc(-c4ccc(S(C)(=O)=O)cc4)oc23)c1F]

2-(3-fluoro-4-(6-(4-(methylsulfonyl)phenyl)furo[3,2-d]pyrimidin-4-yl)pyridin-2-yl)propan-2-ol. Yield 51% as a white solid. 1H NMR (300 MHz, MeOD-d4) δ=9.22 (s, 1H), 8.64 (dd, J=4.9, 1.1 Hz, 1H), 8.37-8.26 (m, 2H), 8.21-8.11 (m, 2H), 7.95 (t, J=4.9 Hz, 1H), 7.83 (s, 1H), 3.21 (s, 3H), 1.75 (d, J=1.4 Hz, 6H); Anal. RP-HPLC tR=0.959 min (method 2, purity 99%); LC-MS ESI: m/z=428.1 [M+H]+ (anal. calcd for C21H19FN3O4S+: m/z=428.1).

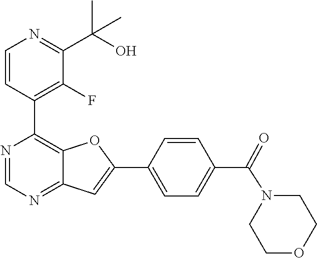 [SMILES: CC(C)(O)c1nccc(-c2ncnc3cc(-c4ccc(C(=O)N5CCOCC5)cc4)oc23)c1F]

(4-(4-(3-fluoro-2-(2-hydroxypropan-2-yl)pyridin-4-yl)furo[3,2-d]pyrimidin-6-yl)phenyl)(morpholino)methanone. Yield 24% as a white solid. 1H NMR (300 MHz, MeOD-d4) δ=9.12 (s, 1H), 8.60 (d, J=4.9 Hz, 1H), 8.10 (s, 2H), 7.89 (dd, J=4.8, 4.8 Hz, 1H), 7.61 (s, 3H), 3.77 (s, 4H), 3.64 (s, 2H), 3.48 (s, 2H), 1.71 (s, 6H); Anal. RP-HPLC tR=0.941 min (method 2, purity 99%); LC-MS ESI: m/z=463.2 [M+H]+ (anal. calcd for C25H24FN4O4+: m/z=463.2).

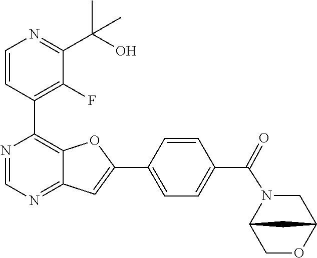 [SMILES: CC(C)(O)c1nccc(-c2ncnc3cc(-c4ccc(C(=O)N5C[C@H]6C[C@@H]5CO6)cc4)oc23)c1F]

((1R,4R)-2-oxa-5-azabicyclo[2.2.1]heptan-5-yl)(4-(4-(3-fluoro-2-(2-hydroxypropan-2-yl)pyridin-4-yl)furo[3,2-d]pyrimidin-6-yl)phenyl)methanone. Yield 27% as a white solid. 1H NMR (300 MHz, MeOD-d4) δ=9.17 (s, 1H), 8.63 (dd, J=4.8, 1.0 Hz, 1H), 8.15 (d, J=9.9, 8.2 Hz, 2H), 7.93 (dd, J=4.8, 4.8 Hz, 1H), 7.80-7.66 (m, 3H), 4.79-4.40 (m, 2H), 4.02 (dd, J=10.1, 7.7 Hz, 1H), 3.94-3.78 (m, 1H), 3.68-3.60 (m, 1H), 3.57-3.35 (m, 1H), 2.12-1.88 (m, 2H), 1.74 (s, 6H); Anal. RP-HPLC tR=0.920 min (method 2, purity 100%); LC-MS ESI: m/z=475.2 [M+H]+ (anal. calcd for C26H24FN4O4+: m/z=475.2).

### BIOLOGICAL ACTIVITY

### Example 2: Impact of Compounds of Plasmodium Growth

Plasmodium PI4K is a recently identified drug target for anti-malaria medication. The compounds according to the present invention were tested for properties relating to the inhibition of Plasmodium growth in which PI4K plays a significant role. Low concentrations of compounds necessary to inhibit growth of Plasmodia are preferred.

In vitro P. falciparum Assay

Compounds according to the present invention were screened against sensitive (NF54) strains of P. falciparum in vitro using the modified [3H]hypoxanthine incorporation assay. (Vennerstrom, J. L.; Arbe-Barnes, S.; Brun, R.; Charman, S. A.; Chiu, F. C. K.; Chollet, J.; Dong, Y.; Dorn, A.; Hunziker, D.; Matile, H.; McIntosh, K.; Padmanilayam, M.; Tomas, J. S.; Scheurer, C.; Scorneaux, B.; Tang, Y.; Urwyler, H.; Wittlin, S. and Charman, W. N. Identification of an Antimalarial Synthetic Trioxolane Drug Development Candidate. Nature, 2004, 430, 900-904.).

Compound concentrations at which growth of P. falciparum was inhibited by 50% compared to untreated controls was measured in nM (IC₅₀).

TABLE 1
Growth inhibition of P. falciparum by compounds
according to the present invention

| Compound | In vitro P. falciparum growth inhibition |
|---|---|
| ID | IC₅₀ (nM) |
| D149 | A |
| D150 | B |
| D183 | A |
| D189 | B |
| D197 | A |
| D203 | A |
| D204 | A |
| D209 | A |
| D210 | A |
| D211 | A |
| D212 | A |
| D213 | B |

IC50: <10 nM = A, 10-50 nM = B, >50 nM = C

These data illustrate the potency of compounds according to the present invention in inhibiting growth of P. falciparum and underline the usefulness as new-generation anti-malaria drugs.

### Example 3: Impact of Compounds on Human PI4K Activity

While Plasmodium PI4K is a desired drug target, many compounds known in the art targeting this enzyme show cross-inhibitory properties with closely related enzymes from other species. In this context, human PI4K can be a problematic off-target as it is involved in essential processes in animals. Therefore, in the context of antimalarials, it is desirable to specifically inhibit Plasmodium PI4K, while only mildly affecting human PI4K.

To further test the impact of compounds according to the present invention on human PI4Kβ, HuPI4Kβ inhibition was tested for selected compounds and contrasted with the results for inhibiting PfNF54 strains as described in the example above.

The lipid kinase reaction is performed by incubating lipid substrate (PI:3PS or PIP2:3PS) with a recombinant enzyme and ATP, and the kinase activity is measured using the ADP-Glo™ Kinase Assay. First, the kinase reaction is terminated, and any ATP remaining after the reaction is depleted, leaving only ADP. Then the Kinase Detection Reagent is added to convert ADP to ATP, which is used in a coupled luciferin/luciferase reaction. The luminescent output is measured and is correlated with kinase activity. The assays can be performed in 96- or 384-well plates and can be used for enzyme characterization, inhibitor screening or compound profiling.

Results measuring the effect of compounds on HuPI4Kβ are reported in table 2 in nM (IC₅₀) describing the concentration necessary to inhibit 50% of the enzyme's activity (HuPI4Kβ IC50 (nM)).

Compound concentrations at which growth of P. falciparum was inhibited by 50% compared to untreated controls was measured in nM (IC₅₀) as described above (PfNF54 IC50 (nM)).

TABLE 2
Inhibitory properties of compounds

| Compound ID | PfNF54 IC₅₀ (nM) | HuPI4Kβ IC₅₀ (nM) |
|---|---|---|
| D149 | A | + |
| D183 | A | + |
| D197 | A | + |
| C74 | B | − |
| C75 | A | − |
| C76 | A | − |
| C77 | B | − |

IC50: <10 nM = A, 10-50 nM = B, >50 nM
IC50: <50 nM = +++, 50-100 nM = ++, 100-500 nM = +, >500 = −

As illustrated in Table 2, several compounds showed high potency to inhibit human PI4K in addition to plasmodium derived PI4K. However, most compounds showed low inhibitory properties in regards to human-derived PI4K, while strongly inhibiting plasmodia growth. These data illustrate, therefore, the excellent use of the compounds according to the present invention for treatment and/or prevention of malaria.

### Example 4: Impact of Compounds on Sensitive Plasmodium Strains

Further compounds according to the present invention have been tested for growth inhibition of NF54 strains in order to evaluate the concentration required to reduce Plasmodium viability and test the efficiency of different compounds according to the present invention in preventing Plasmodium growth.

### In Vitro Antiplasmodial Activity—Assay A

Test samples were screened for in vitro antiplasmodial activity against a chloroquine sensitive (CQS) strain (NF54) of the malaria parasite P. falciparum. Continuous in vitro cultures of asexual erythrocyte stages of P. falciparum were maintained using a modified version of the method of Trager and Jensen (1976).1 Quantitative assessment of antiplasmodial activity in vitro was determined via the parasite lactate dehydrogenase assay using a modified method described by Makler (1993). The test samples were tested in triplicate on two separate occasions. Further dilutions were prepared in complete medium on the day of the experiment. Samples were tested as a suspension if not completely dissolved. Chloroquine and artesunate were used as the reference drugs.

### In Vitro Antiplasmodial Activity—Assay B

Compounds were screened against CQC sensitive strain (NF54) of P. falciparum in vitro as described by Vennerstrom et al. (2004).

A full dose-response were performed starting at a concentration of 3000 nM, which is then serially diluted 2-fold in complete medium to give 10 concentrations; with the lowest concentration being approximately 6 nM. The same dilution techniques were used for all samples. References were tested at a starting concentration of 1000 ng/mL. The highest concentration of solvent to which the parasites were exposed has no measurable effect on the parasite viability (data not shown).

TABLE 3
Inhibitory properties of compounds on Plasmodium growth

| | Assay A | Assay B |
|---|---|---|
| Comp. | NF54 IC50 (nM) | NF54 IC50 (nM) |
| D22 | C | |
| D149 | A | A |
| D150 | C | B |
| D159 | B | A |
| D160 | B | A |
| D161 | A | A |
| D162 | A | |
| D163 | A | |
| D164 | A | |
| D165 | A | |
| D171 | C | |
| D172 | A | A |
| D173 | A | |
| D174 | A | A |
| D175 | A | |
| D176 | A | A |
| D177 | A | A |
| D183 | A | A |
| D191 | A | A |
| D192 | A | A |
| D193 | A | A |
| D194 | A | A |
| D195 | A | A |
| D196 | A | A |
| D197 | A | A |
| D201 | B | B |
| D203 | A | A |
| D204 | | A |
| D207 | A | A |
| D208 | A | A |
| D209 | A | |
| D210 | A | |
| D212 | A | |
| P1 | C | C |
| P2 | | C |
| P3 | | C |
| P4 | | B |
| P5 | | B |
| P6 | | C |
| P7 | | B |
| P8 | | B |
| P9 | | B |
| P10 | | B |
| P11 | | A |
| P12 | | B |
| P13 | C | |
| P14 | | B |
| P15 | | A |
| P16 | | A |
| P17 | C | A |
| P18 | B | A |
| P19 | A | A |
| P20 | A | A |
| P21 | A | A |
| P22 | | A |
| P23 | C | C |
| P24 | C | C |
| P25 | | B |
| P26 | | B |
| P27 | | B |
| P28 | A | A |
| P29 | A | A |
| P30 | A | A |
| P31 | A | A |
| P32 | C | |
| P33 | B | |

IC50: <15 nM = A, 15-50 nM = B, >50 nM = C

The data presented above further provide clear evidence for the use of the compounds according to the present invention for the treatment and/or prevention of malaria. Concentrations as low as <1 nM were able to inhibit Plasmodium growth by 50%.

### Example 5: In Vitro P. vivax Liver-Stage Assay

Compound efficacy against P. vivax liver-stage schizonts and hypnozoites is evaluated in infected primary human hepatocytes (PHHs). Compound screening is performed in 384-well plates, in a 12-point dose response, from 50 μM. All compounds are tested in radical cure mode (RCM).

PHHs are seeded onto 384-well plates two days prior to P. vivax sporozoite infection. In RCM, compounds are added to the culture for three days, from day 5 post infection, in duplicate wells. Nigericin and a PI4K inhibitor (KDU691) are used as positive controls in each assay, while solvent (DMSO) without added compound serves as a negative control. Daily medium changes are performed, and the culture is fixed on day twelve post infection to ensure complete parasite clearance. Fixed cells are permeabilized and stained with an anti-UIS4 primary antibody and a fluorescent secondary antibody. Liver-stage schizonts and hypnozoites are then quantified by High Content Imaging.

Roth, A., et al. A comprehensive model for assessment of liver stage therapies targeting Plasmodium vivax and Plasmodium falciparum. Nat. Commun. 2018, 9(1), 1837. doi.org/10.1038/s41467-018-04221-9.

TABLE 4
In vitro P. vivax liver-stage assay data

| | Pv | |
|---|---|---|
| Compound | Hypnozoite | Pv Schizont |
| ID/Structure | IC₅₀ (nM) | IC₅₀ (nM) |
| D149 | A | A |
| D183 | A | A |
| D201 | B | A |
| D203 | B | A |
| D213 | C | A |

IC50: <15 nM = A, 15-50 nM = B, >50 nM = C

1. A compound according to formula (I), 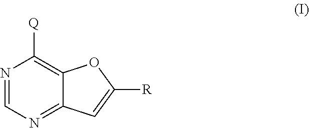
or a pharmaceutically acceptable solvate, salt, tautomer or stereoisomer thereof, wherein:
R denotes AR1 or HT1;
AR1 denotes phenyl, which is unsubstituted or substituted by
1, 2 or 3 substituents independently selected from the group consisting of Alk2, OAlk2, Hal, Cyc, CN and/or NO₂; and/or
a substituent selected from a group comprising of A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
HT1 denotes a mono- or bicyclic saturated, unsaturated or aromatic heterocycle with 3 to 9 carbon atoms and 1, 2, 3 or 4 N, O and/or S atoms, wherein said heterocycle is unsubstituted or substituted by:
1, 2 or 3 substituents independently selected from the group consisting of Alk2, OAlk2, Hal, Cyc, CN, ═O and/or NO₂; and/or
a substituent selected from the group consisting of A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyC, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCycl, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}) (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
Q denotes a structure according to formula (II) 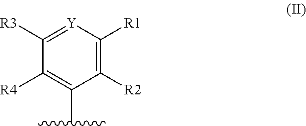
R¹ denotes AR2 or HT2;
R², R³ and R⁴ denote, independently from each other, H, Hal or CAlk2;
Y denotes CH, CHal, CAlk2, CCHal3 or N;
AR2 denotes phenyl, which is unsubstituted or substituted by
1, 2 or 3 substituents independently selected from the group consisting of Alk2, OAlk2, Hal, Cyc, CN and/or NO₂; and/or
a substituent selected from a group consisting of A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyc, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
HT2 denotes a mono- or bicyclic saturated, unsaturated or aromatic heterocycle with 3 to 9 carbon atoms and 1, 2, 3 or 4 N, O and/or S atoms, wherein said heterocycle is unsubstituted or substituted by
1, 2 or 3 substituents independently selected from the group consisting of Alk2, OAlk2, Hal, Cyc, CN, ═O and/or NO₂; and/or
a substituent selected from a group consisting of A, NH₂, OH, (CR^{a}R^{b})ₙHetCyc1, (CR^{a}R^{b})ₙHetAr1, (CR^{a}R^{b})ₙAryl, (CR^{a}R^{b})ₙCO(R^{a}R^{b})ₘHetCyc1, (CR^{a}R^{b})ₙCO(R^{a}R^{b})ₘHetAr1, (CR^{a}R^{b})ₙCO(R^{a}R^{b})ₘAryl, (CR^{a}R^{b})ₙCOCyc, (CR^{a}R^{b})ₙCOA, (CR^{a}R^{b})ₙCONA₂, (CR^{a}R^{b})ₙCONH₂, (CR^{a}R^{b})ₙCONHA, (CR^{a}R^{b})ₙCONR^{Cyc3}R^{Cyc4}, (CR^{a}R^{b})ₙCONH(CR^{a}R^{b})ₘHetCyc1, (CR^{a}R^{b})ₙCONH(CR^{a}R^{b})ₘHetAr1, (CR^{a}R^{b})ₙCONH(R^{a}R^{b})ₘAryl, (CR^{a}R^{b})ₙCONHCyc, (CR^{a}R^{b})ₙCOOA, (CR^{a}R^{b})ₙCOOH, (CR^{a}R^{b})ₙCOO(CR^{a}R^{b})ₘHetCyc1, (CR^{a}R^{b})ₙCOO(CR^{a}R^{b})ₘHetAr1, (CR^{a}R^{b})ₙCOO(R^{a}R^{b})ₘAryl, (CR^{a}R^{b})ₙCOOCyC, (CR^{a}R^{b})ₙNHCO(R^{a}R^{b})ₘHetCyc1, (CR^{a}R^{b})ₙNHCO(R^{a}R^{b})ₘHetAr1, (CR^{a}R^{b})ₙNHCO(R^{a}R^{b})ₘAryl, (CR^{a}R^{b})ₙNHCOCyc, (CR^{a}R^{b})ₙNHCOA, (CR^{a}R^{b})ₙS(R^{a}R^{b})ₘHetCyc1, (CR^{a}R^{b})ₙS(R^{a}R^{b})ₘHetAr1, (CR^{a}R^{b})ₙS(R^{a}R^{b})ₘAryl, (CR^{a}R^{b})ₙSA, (CR^{a}R^{b})ₙSO(R^{a}R^{b})ₘHetCyc1, (CR^{a}R^{b})ₙSO(R^{a}R^{b})ₘHetAr1, (CR^{a}R^{b})ₙSO(R^{a}R^{b})ₘAryl, (CR^{a}R^{b})ₙSOA, (CR^{a}R^{b})ₙSO₂(R^{a}R^{b})ₘHetCyc1, (CR^{a}R^{b})ₙSO₂(R^{a}R^{b})ₘHetAr1, (CR^{a}R^{b})ₙSO₂(R^{a}R^{b})ₘAryl, (CR^{a}R^{b})ₙSO₂CyC, (CR^{a}R^{b})ₙSO₂A, (CR^{a}R^{b})ₙSOA(NH), (CR^{a}R^{b})ₙSOCyc(NH), (CR^{a}R^{b})ₙSOAryl(NH), (CR^{a}R^{b})ₙSOHetCyc1(NH), (CR^{a}R^{b})ₙSOHetAr1(NH), (CR^{a}R^{b})ₙSOA(NA), (CR^{a}R^{b})ₙSOR^{Cyc1}(NR^{Cyc2}), (CR^{a}R^{b})ₙSOCyc(NA), (CR^{a}R^{b})ₙSOAryl(NA), (CR^{a}R^{b})ₙSOHetCyc1(NA), (CR^{a}R^{b})ₙSOHetAr1(NA), (CR^{a}R^{b})ₙSOA(NCyc), (CR^{a}R^{b})ₙSOCyc(NCyc), (CR^{a}R^{b})ₙSOAryl(NCyc), (CR^{a}R^{b})ₙSOHetCyc1(NCyc), (CR^{a}R^{b})ₙSOHetAr1(NCyc), (CR^{a}R^{b})ₙSO₂NA₂, (CR^{a}R^{b})ₙSO₂NH₂, (CR^{a}R^{b})ₙSO₂NHA and (CR^{a}R^{b})ₙPOA₂;
A denotes linear or branched alkyl having 1, 2, 3, 4, 5, or 6 carbon atoms, wherein:
one or two non-adjacent CH₂ groups may be replaced by O, NAlk2 or NH; and/or
1, 2, 3, 4 or 5 hydrogens may be replaced by Hal; and/or
one hydrogen may be replaced by OH or NH₂ or a cyclic alkyl having 3, 4, 5 or 6 carbon atoms, which is mono- di or trisubstituted by Hal, OH, Alk2, NHAlk2, N(Alk2)₂ and/or NH₂;
Alk1 denotes linear or branched alkyl having 1, 2, 3, 4, 5, or 6 carbon atoms, wherein
one or two CH₂ groups may be replaced by O, NAlk2 or NH; and/or
one hydrogen may be replaced by OH, NHAlk2, N(Alk2)₂ or NH₂; and/or
1, 2, 3, 4 or 5 hydrogens may be replaced by Hal;
Alk2 denotes linear or branched alkyl having 1 to 6 carbon atoms, wherein 1, 2, 3, 4 or 5 hydrogens may be replaced by Hal;
Aryl denotes phenyl, which is unsubstituted or mono-, di- or trisubstituted Hal, Alk2, OAlk2, OH, NH₂ or Cyc;
HetCyc1 denotes a mono- or bicyclic optionally bridged saturated or unsaturated 4- to 10-membered heterocycle having 1 or 2 heteroatoms selected from N, O, S and/or Si, wherein said heterocycle may be unsubstituted or mono- or disubstituted by Hal, OH, A, SO₂Alk2 and/or ═O;
Cyc denotes cyclic alkyl having 3 to 6 carbon atoms, wherein 1, 2 or 3 hydrogens may be replaced by Hal and 1 additional hydrogen may be replaced by Alk2, NH₂ and/or OH;
Hal denotes F or Cl;
HetAr1 denotes a mono- or bicyclic aromatic 4- to 12-membered heterocycle having 1, 2, 3 or 4 N, O and/or S atoms, said heterocycle being unsubstituted or mono- or disubstituted Hal, Alk2, SOAlk2, SO₂Alk2, OH or NH₂;
R^{a} and R^{b} denote each, independently from each other, H, Alk2 or Cyc; or
R^{a} and R^{b} together represent —(CH₂)ₓ— with x=2, 3, 4 or 5, thus forming together with the carbon atom they are attached to a (3-, 4-, 5- or 6-membered) cycloalkyl ring;
R^{Cyc1} and R^{Cyc2} together form —(CH₂)ₓ— with x=3 or 4, thus forming together with the atoms they are attached to a (5- or 6-membered) ring, wherein 1, or 2H atoms, in —(CH₂)ₓ— may be independently replaced by Hal or Alk1;
R^{Cyc3} and R^{Cyc4} together form —(CH₂)ₓ— with x=3, 4 or 5, thus forming together with the nitrogen atom they are attached to a (4-, 5- or 6-membered) ring, wherein 1, or 2H atoms, in —(CH₂)ₓ— may be independently replaced by Hal or Alk1;
n denotes 0, 1 or 2; and
m denotes 0 or 1.

2. The compound according to claim 1, wherein R denotes a structure according to formula (IV), (V), (Va) or (VI) 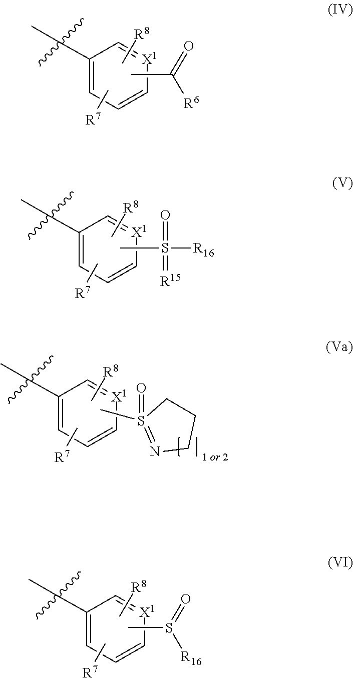
wherein
R⁶ denotes OH, A or Cyc or a substituent according to formula (VII) to (X) 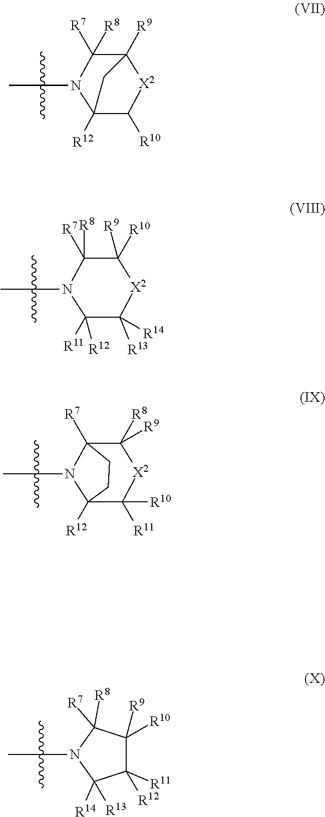
wherein
R⁷, R⁸, R⁹, R¹⁰, R¹¹, R¹², R¹³ and R¹⁴ denote each, independently from each other, H, OH, Hal, CH₃, C₂H₅, CHal₃, OCH₃, OCHal₃, OCHal₂, OCH₂Hal, CH₂Hal and/or CHHal₂;
R¹⁵ denotes NR¹⁷ or O;
R¹⁶ denotes A or Cyc;
R¹⁷ denotes H, Alk1 or cyclic alkyl having 3 to 6 carbon atoms, wherein 1, 2 or 3 hydrogens of said cyclic alkyl group may be replaced by Hal;
X¹ denotes N or CH; and
X² denotes NH, NAlk1 or O.

3. The compound according to claim 1, wherein Q denotes a structure according to formula (XI) 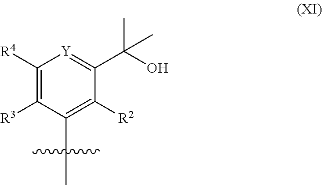
wherein one or two of the residues R², R³ and R⁴ independently represent, Hal, CH₃, CHal₃, OCH₃, OCHal₃, OCHHal₂, OCH₂Hal, CH₂Hal and/or CHHal₂ and the remaining residue(s) represent H.

4. The compound according to claim 1, selected from the group consisting of:

| Comp. | Name |
|---|---|
| C74 | (4-{4-[2-(1-Hydroxy-1-methyl-ethyl)-pyridin-4-yl]-furo[3,2-d]pyrimidin-6- |
| | yl}-phenyl)-morpholin-4-yl-methanone; |
| C75 | (4-{4-[2-(1-Hydroxy-1-methyl-ethyl)-pyridin-4-yl]-furo[3,2-d]pyrimidin-6- |
| | yl}-phenyl)-((R)-3-methyl-morpholin-4-yl)-methanone; |
| C76 | 2-[4-(6-{4-[(2R,6S)-2,6-dimethylmorpholine-4-carbonyl]phenyl}furo[3,2- |
| | d]pyrimidin-4-yl)pyridin-2-yl]propan-2-ol; |
| C77 | 2-(4-{6-[4-(4-methylpiperazine-1-carbonyl)phenyl]furo[3,2-d]pyrimidin-4- |
| | yl}pyridin-2-yl)propan-2-ol; |
| D22 | 5-(4-(3-(cyclopropylsulfonyl)phenyl)furo[3,2-d]pyrimidin-6-yl)-1- |
| | methylpyridin-2(1H)-one; |
| D149 | 2-(2-fluoro-3-(6-(4-(methylsulfonyl)phenyl)furo[3,2-d]pyrimidin-4- |
| | yl)phenyl)propan-2-ol; |
| D150 | 2-(6-fluoro-4-(6-(4-(methylsulfonyl)phenyl)furo[3,2-d]pyrimidin-4- |
| | yl)pyridin-2-yl)propan-2-ol; |
| D159 | (S)-(4-(4-(2-(2-hydroxypropan-2-yl)pyridin-4-yl)furo[3,2-d]pyrimidin-6- |
| | yl)phenyl)(3-methylmorpholino)methanone; |
| D160 | (R)-(4-(4-(2-(2-hydroxypropan-2-yl)pyridin-4-yl)furo[3,2-d]pyrimidin-6- |
| | yl)phenyl)(3-methylmorpholino)methanone; |
| D161 | (2,6-dimethylmorpholino)(4-(4-(2-(2-hydroxypropan-2-yl)pyridin-4- |
| | yl)furo[3,2-d]pyrimidin-6-yl)phenyl)methanone; |
| D162 | tert-butyl 4-(4-(2-(2-hydroxypropan-2-yl)pyridin-4-yl)furo[3,2- |
| | d]pyrimidin-6-yl)benzoate; |
| D163 | methyl 3-(4-(2-(2-hydroxypropan-2-yl)pyridin-4-yl)furo[3,2-d]pyrimidin- |
| | 6-yl)benzoate; |
| D164 | 3-(4-(2-(2-hydroxypropan-2-yl)pyridin-4-yl)furo[3,2-d]pyrimidin-6- |
| | yl)benzoic acid; |
| D165 | (4-(4-(3-fluoro-5-(2-hydroxypropan-2-yl)phenyl)furo[3,2-d]pyrimidin-6- |
| | yl)phenyl)(morpholino)methanone; |
| D171 | (3,5-dimethylmorpholino)(4-(4-(2-(2-hydroxypropan-2-yl)pyridin-4- |
| | yl)furo[3,2-d]pyrimidin-6-yl)phenyl)methanone; |
| D172 | ((1R,4R)-2-oxa-5-azabicyclo[2.2.1]heptan-5-yl)(4-(4-(3-(2-hydroxypropan- |
| | 2-yl)phenyl)furo[3,2-d]pyrimidin-6-yl)phenyl)methanone; |
| D173 | ((1R,4R)-2-oxa-5-azabicyclo[2.2.1]heptan-5-yl)(4-(4-(3-fluoro-5-(2- |
| | hydroxypropan-2-yl)phenyl)furo[3,2-d]pyrimidin-6-yl)phenyl)methanone; |
| D174 | ((1S,4S)-2-oxa-5-azabicyclo[2.2.1]heptan-5-yl)(4-(4-(3-(2-hydroxypropan- |
| | 2-yl)phenyl)furo[3,2-d]pyrimidin-6-yl)phenyl)methanone; |
| D175 | ((1S,4S)-2-oxa-5-azabicyclo[2.2.1]heptan-5-yl)(4-(4-(3-fluoro-5-(2- |
| | hydroxypropan-2-yl)phenyl)furo[3,2-d]pyrimidin-6-yl)phenyl)methanone; |
| D176 | ((1S,4S)-2-oxa-5-azabicyclo[2.2.1]heptan-5-yl)(4-(4-(2-(2-hydroxypropan- |
| | 2-yl)pyridin-4-yl)furo[3,2-d]pyrimidin-6-yl)phenyl)methanone; |
| D177 | ((1R,4R)-2-oxa-5-azabicyclo[2.2.1]heptan-5-yl)(4-(4-(2-(2-hydroxypropan- |
| | 2-yl)pyridin-4-yl)furo[3,2-d]pyrimidin-6-yl)phenyl)methanone; |
| D183 | (4-(4-(2-fluoro-3-(2-hydroxypropan-2-yl)phenyl)furo[3,2-d]pyrimidin-6- |
| | yl)phenyl)(morpholino)methanone; |
| D191 | (S)-(4-(4-(2-fluoro-3-(2-hydroxypropan-2-yl)phenyl)furo[3,2-d]pyrimidin- |
| | 6-yl)phenyl)(2-methylmorpholino)methanone; |
| D192 | (R)-(4-(4-(2-fluoro-3-(2-hydroxypropan-2-yl)phenyl)furo[3,2-d]pyrimidin- |
| | 6-yl)phenyl)(2-methylmorpholino)methanone; |
| D193 | ((2R,6R)-2,6-dimethylmorpholino)(4-(4-(2-fluoro-3-(2-hydroxypropan-2- |
| | yl)phenyl)furo[3,2-d]pyrimidin-6-yl)phenyl)methanone; |
| D194 | ((2S,6S)-2,6-dimethylmorpholino)(4-(4-(2-fluoro-3-(2-hydroxypropan-2- |
| | yl)phenyl)furo[3,2-d]pyrimidin-6-yl)phenyl)methanone; |
| D195 | (S)-(4-(4-(2-fluoro-3-(2-hydroxypropan-2-yl)phenyl)furo[3,2-d]pyrimidin- |
| | 6-yl)phenyl)(3-methylmorpholino)methanone; |
| D196 | (R)-(4-(4-(2-fluoro-3-(2-hydroxypropan-2-yl)phenyl)furo[3,2-d]pyrimidin- |
| | 6-yl)phenyl)(3-methylmorpholino)methanone; |
| D197 | ((1S,4S)-2-oxa-5-azabicyclo[2.2.1]heptan-5-yl)(4-(4-(2-fluoro-3-(2- |
| | hydroxypropan-2-yl)phenyl)furo[3,2-d]pyrimidin-6-yl)phenyl)methanone; |
| D201 | 4-(4-(2-fluoro-3-(2-hydroxypropan-2-yl)phenyl)furo[3,2-d]pyrimidin-6- |
| | yl)benzoic acid; |
| D203 | (4-(4-(2-fluoro-3-(2-hydroxypropan-2-yl)phenyl)furo[3,2-d]pyrimidin-6- |
| | yl)phenyl)(imino)(methyl)-λ6-sulfanone; |
| D204 | (4-(4-(2-fluoro-3-(2-hydroxypropan-2-yl)phenyl)furo[3,2-d]pyrimidin-6- |
| | yl)phenyl)(4-methylpiperazin-1-yl)methanone; |
| D207 | (4-(4-(3-fluoro-2-(2-hydroxypropan-2-yl)pyridin-4-yl)furo[3,2- |
| | d]pyrimidin-6-yl)phenyl)(morpholino)methanone; |
| D208 | ((1R,4R)-2-oxa-5-azabicyclo[2.2.1]heptan-5-yl)(4-(4-(3-fluoro-2-(2- |
| | hydroxypropan-2-yl)pyridin-4-yl)furo[3,2-d]pyrimidin-6- |
| | yl)phenyl)methanone; |
| D209 | (S)-(4-(4-(2-fluoro-3-(2-hydroxypropan-2-yl)phenyl)furo[3,2-d]pyrimidin- |
| | 6-yl)phenyl)(imino)(methyl)-λ⁶-sulfanone; |
| D210 | (R)-(4-(4-(2-fluoro-3-(2-hydroxypropan-2-yl)phenyl)furo[3,2-d]pyrimidin- |
| | 6-yl)phenyl)(imino)(methyl)-λ⁶-sulfanone; |
| D211 | 1-(4-(4-(2-fluoro-3-(2-hydroxypropan-2-yl)phenyl)furo[3,2-d]pyrimidin-6- |
| | yl)phenyl)-3,4,5,6-tetrahydro-1,2-thiazine 1-oxide; |
| D212 | 1-(4-(4-(2-fluoro-3-(2-hydroxypropan-2-yl)phenyl)furo[3,2-d]pyrimidin-6- |
| | yl)phenyl)-4,5-dihydro-3H-isothiazole 1-oxide; |
| D213 | 2-(4-(6-(4-(methylsulfonyl)phenyl)furo[3,2-d]pyrimidin-4-yl)pyridin-2- |
| | yl)propan-2-ol; |
| P1 | 1-(4-(6-(4-(methylsulfonyl)phenyl)furo[3,2-d\|pyrimidin-4-yl)pyridin-2- |
| | yl)azetidin-3-ol; |
| P2 | 1-(5-(4-(3-(cyclopropylsulfonyl)phenyl)furo[3,2-d]pyrimidin-6-yl)pyridin- |
| | 2-yl)ethan-1-one; |
| P3 | ((1R,4R)-2-oxa-5-azabicyclo[2.2. 1]heptan-5-yl)(3-(6-(4- |
| | (methylsulfonyl)phenyl)furo[3,2-d]pyrimidin-4-yl)phenyl)methanone; |
| P4 | ((1R,4R)-2-oxa-5-azabicyclo[2.2.1]heptan-5-yl)(2-fluoro-3-(6-(4- |
| | (methylsulfonyl)phenyl)furo[3,2-d]pyrimidin-4-yl)phenyl)methanone; |
| P5 | ((1S,4S)-2-oxa-5-azabicyclo[2.2.1]heptan-5-yl)(2-fluoro-3-(6-(4- |
| | (methylsulfonyl)phenyl)furo[3,2-d]pyrimidin-4-yl)phenyl)methanone; |
| P6 | 2-methyl-1-(4-(6-(4-(methylsulfonyl)phenyl)furo[3,2-d]pyrimidin-4- |
| | yl)pyridin-2-yl)propan-2-ol; |
| P7 | 2-(4-(6-(4-(methylsulfonyl)phenyl)furo[3,2-d]pyrimidin-4-yl)pyridin-2- |
| | yl)propan-2-ol; |
| P8 | 4-(4-(2-(2-hydroxypropan-2-yl)pyridin-4-yl)furo[3,2-d]pyrimidin-6-yl)- |
| | N,N-dimethylbenzamide; |
| P9 | 4-(4-(3-(2-hydroxypropan-2-yl)phenyl)furo[3,2-d]pyrimidin-6-yl)-N,N- |
| | dimethylbenzamide; |
| P10 | 4-(4-(2-(2-hydroxypropan-2-yl)pyridin-4-yl)furo[3,2-d]pyrimidin-6- |
| | yl)benzoic acid; |
| P11 | (4-(4-(2-(2-hydroxypropan-2-yl)pyridin-4-yl)furo[3,2-d]pyrimidin-6- |
| | yl)phenyl)(morpholino)methanone; |
| P12 | (3,3-difluoroazetidin-1-yl)(4-(4-(2-(2-hydroxypropan-2-yl)pyridin-4- |
| | yl)furo[3,2-d]pyrimidin-6-yl)phenyl)methanone; |
| P13 | (4-(4-(3-(2-hydroxypropan-2-yl)phenyl)furo[3,2-d]pyrimidin-6- |
| | yl)phenyl)(morpholino)methanone; |
| P14 | (4-(4-(2-fluoro-6-(2-hydroxypropan-2-yl)pyridin-4-yl)furo[3,2- |
| | d]pyrimidin-6-yl)phenyl)(morpholino)methanone; |
| P15 | ((2R,6R)-2,6-dimethylmorpholino)(4-(4-(2-(2-hydroxypropan-2-yl)pyridin- |
| | 4-yl)furo[3,2-d]pyrimidin-6-yl)phenyl)methanone; |
| P16 | ((2S,6S)-2,6-dimethylmorpholino)(4-(4-(2-(2-hydroxypropan-2-yl)pyridin- |
| | 4-yl)furo[3,2-d]pyrimidin-6-yl)phenyl)methanone; |
| P17 | ((2S,6R)-2,6-dimethylmorpholino)(4-(4-(2-(2-hydroxypropan-2-yl)pyridin- |
| | 4-yl)furo[3,2-d]pyrimidin-6-yl)phenyl)methanone; |
| P18 | (S)-(4-(4-(2-(2-hydroxypropan-2-yl)pyridin-4-yl)furo[3,2-d]pyrimidin-6- |
| | yl)phenyl)(2-methylmorpholino)methanone; |
| P19 | (R)-(4-(4-(2-(2-hydroxypropan-2-yl)pyridin-4-yl)furo[3,2-d]pyrimidin-6- |
| | yl)phenyl)(2-methylmorpholino)methanone; |
| P20 | ((S8,6R)-2,6-dimethylmorpholino)(4-(4-(2-fluoro-3-(2-hydroxypropan-2- |
| | yl)phenyl)furo[3,2-d]pyrimidin-6-yl)phenyl)methanone; |
| P21 | ((1R,4R)-2-oxa-5-azabicyclo[2.2.1]heptan-5-yl)(4-(4-(2-fluoro-3-(2- |
| | hydroxypropan-2-yl)phenyl)furo[3,2-d]pyrimidin-6-yl)phenyl)methanone; |
| P22 | (4-(4-(2-(2-hydroxypropan-2-yl)pyridin-4-yl)furo[3,2-d]pyrimidin-6- |
| | yl)phenyl)(4-methylpiperazin-1-yl)methanone; |
| P23 | ((1S,4S)-2-oxa-5-azabicyclo[2.2.1]heptan-5-yl)(1-(4-(4-(2-(2- |
| | hydroxypropan-2-yl)pyridin-4-yl)furo[3,2-d]pyrimidin-6- |
| | yl)phenyl)cyclopropyl)methanone; |
| P24 | ((1R,4R)-2-oxa-5-azabicyclo[2.2.1]heptan-5-yl)(1-(4-(4-(2-(2- |
| | hydroxypropan-2-yl)pyridin-4-yl)furo[3,2-d]pyrimidin-6- |
| | yl)phenyl)cyclopropyl)methanone; |
| P25 | (4-(4-(2-(2-hydroxypropan-2-yl)pyridin-4-yl)furo[3,2-d]pyrimidin-6- |
| | yl)phenyl)(imino)(methyl)-26-sulfanone; |
| P26 | (S)-(4-(4-(2-(2-hydroxypropan-2-yl)pyridin-4-yl)furo[3,2-d]pyrimidin-6- |
| | yl)phenyl)(imino)(methyl)-26-sulfanone; |
| P27 | (R)-(4-(4-(2-(2-hydroxypropan-2-yl)pyridin-4-yl)furo[3,2-d]pyrimidin-6- |
| | yl)phenyl)(imino)(methyl)-λ6-sulfanone; |
| P28 | (4-(4-(2-fluoro-3-(2-hydroxypropan-2-yl)phenyl)furo[3,2-d]pyrimidin-6- |
| | yl)phenyl)(imino)(methyl)-26-sulfanone; |
| P29 | (S)-(4-(4-(2-fluoro-3-(2-hydroxypropan-2-yl)phenyl)furo[3,2-d]pyrimidin- |
| | 6-yl)phenyl)(imino)(methyl)-26-sulfanone; |
| P30 | (R)-(4-(4-(2-fluoro-3-(2-hydroxypropan-2-yl)phenyl)furo[3,2-d]pyrimidin- |
| | 6-yl)phenyl)(imino)(methyl)-26-sulfanone; |
| P31 | 2-(3-fluoro-4-(6-(4-(methylsulfonyl)phenyl)furo[3,2-d]pyrimidin-4- |
| | yl)pyridin-2-yl)propan-2-ol; |
| P32 | (4-(4-(4-fluoro-3-(2-hydroxypropan-2-yl)phenyl)furo[3,2-d]pyrimidin-6- |
| | yl)phenyl)(morpholino)methanone; and |
| P33 | 4-(4-(2-fluoro-3-(2-hydroxypropan-2-yl)phenyl)furo[3,2-d]pyrimidin-6- |
| | yl)benzenesulfonamide[[.]] |

and pharmaceutically acceptable solvates, salts, tautomers and stereoisomers thereof, and mixtures thereof in all ratios.

5. A method, comprising:
inhibiting of PI4K with the compound according to claim 1.

6. A method, comprising:
treating and/or preventing of a PI4K related disorder with the compound according to claim 1.

7-15. (canceled)

16. The compound according to claim 1 wherein R has a structure of formula (IV): 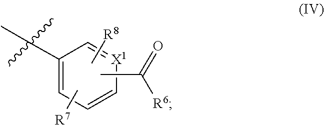
wherein
R⁶ is Alk1, Alk2, —OH, —CH₃, —OCH₃, —OC(CH₃)₃, —N(CH₃)₂, 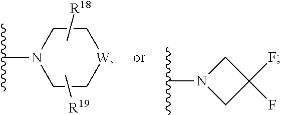
W is O, NR¹⁸, or CR¹⁸R¹⁹;
R⁷, R⁸ is each independently selected from —H or Hal;
R¹⁸, R¹⁹ is each independently selected from —H, —CH₃, or Alk1;
X¹ is CR⁷ or N; and
wherein Q is formula (II) and
R¹ is H, Hal, 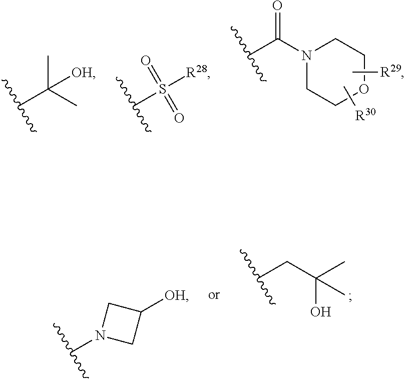
R², R³, R⁴ is each independently selected from H or Hal;
R²⁸ is Alk1, Alk2, —NH₂, 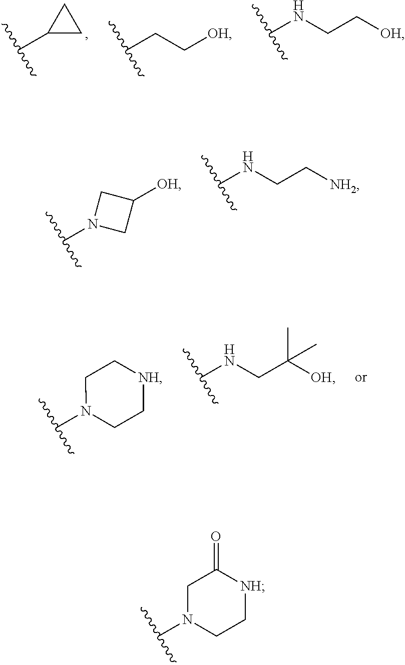
R²⁹, R³⁰ is each independently selected from —H or —CH₃;
Y is N, CH, or CHal.

17. The compound according to claim 1, wherein R has a structure of formula (V): 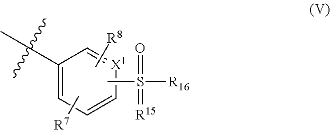
X¹ is CR⁷ or N;
R⁷, R⁸ is each independently selected from H, Hal, or CHal₃;
R¹⁵ is O or NH;
R¹⁶ is H, —CH₃, —NH₂, —N(CH₃)₂ or Alk1; and
wherein Q is formula (II) and
R¹ is H, Hal, 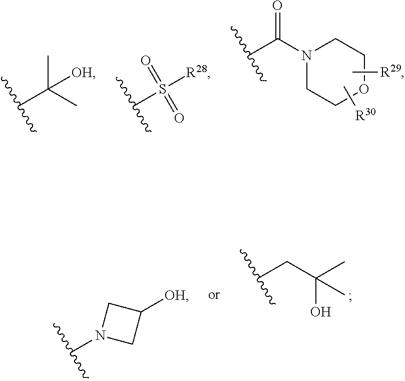
R¹, R³, R⁴ is each independently selected from H or Hal;
R²⁸ is Alk1, Alk2, —NH₂, 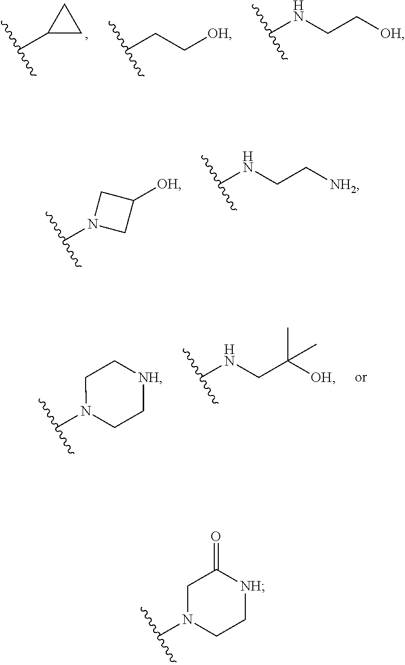
R²⁹, R³⁰ is each independently selected from H or CH₃;
Y is N, CH, or (Hal.

18. The compound according to claim 1, wherein R has a structure of 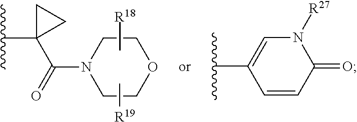
R¹⁸, R¹⁹ is each independently selected from H, —CH₃, or Alk1; and
R²⁷ is —CH₃ or —C((CH₃)₂OH);
wherein Q is formula (II) and
R¹ is H, Hal, 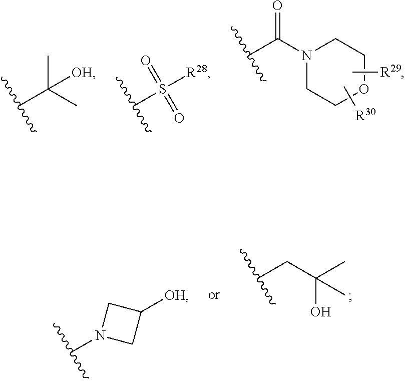
R², R³, R⁴ is each independently selected from H or Hal;
R²⁸ is Alk1, Alk2, —NH₂, 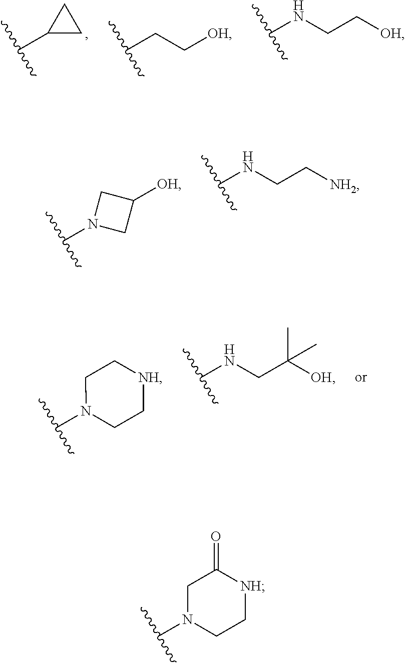
R²⁹, R³⁰ is each independently selected from H or CH₃;
Y is N, CH or CHal.

19. The compound according to claim 16, wherein
R⁶ is —CH₃, —OH, —N(CH₃)₂; 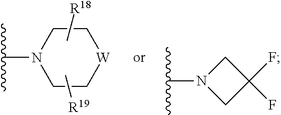
W is O;
R⁷, R⁸ is H;
R¹⁸, R¹⁹ is each independently selected from —H or —CH₃;
X¹ is CH or N.

20. The compound according to claim 19, wherein
R⁶ is 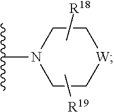
and
R¹ is

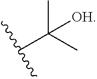 [SMILES: *C(C)(C)O]

21. The compound according to claim 17, wherein
R¹ is selected from H, Hal, or 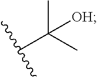 [SMILES: *C(C)(C)O]
R², R³, R⁴, R⁷, R⁸ is each independently selected from H or Hal;
R¹⁶ is —CH₃ or Alk1;
X¹ is CH; and
Y is CH or CHal.

22. The compound according to claim 21, wherein
R¹ is 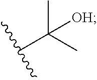 [SMILES: *C(C)(C)O]
R², R⁷, R⁸ is each independently selected from H or Hal;
R³, R⁴ is H;
R¹⁵ is NH;
R¹⁶ is —CH₃ or Alk1;
X¹ is CH; and
Y is CH.

23. The compound of claim 22, wherein the SOR¹⁵R¹⁶ group of formula V is ortho to the linkage of formula I.

24. The compound of claim 23, wherein R³ and R⁴ are H.

25. The compound according to claim 24, wherein X¹ is CH.

26. The compound according to claim 25, wherein
R¹ is 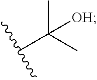 [SMILES: *C(C)(C)O]
R², R⁷, R⁸ is each independently selected from H or Hal;
R³, R⁴ is H;
R¹⁵ is NH;
R¹⁶ is —CH₃;
X¹ is CH; and
Y is CH.

27. The compound according to claim 26, wherein the compound is

| | |
|---|---|
| D203 | (4-(4-(2-fluoro-3-(2-hydroxypropan-2-yl)phenyl)furo[3,2- |
| | d]pyrimidin-6-yl)phenyl)(imino)(methyl)-λ6-sulfanone. |

28. The compound according to claim 26, wherein the compound is

| | |
|---|---|
| D209 | (S)-(4-(4-(2-fluoro-3-(2-hydroxypropan-2-yl)phenyl)furo[3,2- |
| | d]pyrimidin-6-yl)phenyl)(imino)(methyl)-λ⁶-sulfanone. |

29. The compound according to claim 26, wherein the compound is

| | |
|---|---|
| D210 | (R)-(4-(4-(2-fluoro-3-(2-hydroxypropan-2-yl)phenyl)furo[3,2- |
| | d]pyrimidin-6-yl)phenyl)(imino)(methyl)-λ⁶-sulfanone. |
